(19) United States
(12) Patent Application Publication
(10) Pub. No.: US 2004/0096901 A1
(43) Pub. Date: May 20, 2004

(54) Therapeutic binding molecules

(76) Inventors: Gregorio Aversa, Vienna (AT); Frank Kolbinger, Neuenberg (DE); Jos?eacute; M Carballido Herrera, Vienna (AT); Andr?aacute;s Aszódi, Vienna (AT); Jos?eacute; W Saldanha, London (GB); Bruce M Hall, Strathfield NSW (AU)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/467,546

(22) PCT Filed: Feb. 11, 2002
(86) PCT No.: PCT/EP02/01420

(30) Foreign Application Priority Data
Dec. 2, 2001 (GB) 0103389.3

(51) Int. Cl. C12Q 1/68; G01N 33/53; C07K 16/44; C07K 16/28

(52) U.S. Cl. 435/7.1; 530/387.3; 530/388.15; 530/388.22

(57) ABSTRACT

A molecule comprising at least one antigen binding site, comprising in sequence the hypervariable regions CDR1, CDR2 and CDR3, said CDR1 having the amino acid sequence Asn-Tyr-Ile-Ile-His (NYTH), said CDR2 having the amino acid sequence Tyr-Phe-Asn-Pro-Tyr-Asn-His-Gly-Thr-Lys-Tyr-Asn-Glu-Lys-Phe-Lys-Gly (YFYNHGTKYNEKFKG) and said CDR3 having the amino acid seuqnce Ser-Gly-Pro-Tyr-Ala-Trp-Phe-Asp-Thr (SGPYAWFDT); e.g. further comprising in sequence the hypervariable regions CDR1′, CDR2′ and CDR3′, CDR1′ having the amino acid sequence Arg-Ala-Ser-Gln-Asn-Ile-Gly-Thr-Ser-Ile-Gln 8RASQNIGTSIQ), CDR′, having the amino acid sequence Ser-Ser-Ser-Glu-Ser-Ile-Ser (SSSESIS) and CDR3′ having the amino acid sequence Gln-Gln-Ser-Asn-Thr-Trp-Pro-Phe-Thr (QQSNTWPFT), e.g. a chmieric or humanised antibody, useful as a pharmaceutical.

### FIELD OF THE INVENTION

The present invention relates to organic compounds, such as to binding molecules against CD45 antigen isoforms, such as for example monoclonal antibodies (mAbs).

### BACKGROUND OF THE INVENTION

One approach in the treatment of a variety of diseases is to achieve the elimination or the inactivation of pathogenic leukocytes and the potential for induction of tolerance to inactivate pathological immune responses.

Organ, cell and tissue transplant rejection and the various autoimmune diseases are thought to be primarily the result of T-cell mediated immune response triggered by helper T-cells which are capable of recognizing specific antigens which are captured, processed and presented to the helper T cells by antigen presenting cell (APC) such as macrophages and dendritic cells, in the form of an antigen-MHC complex, i.e. the helper T-cell when recognizing specific antigens is stimulated to produce cytokines such as IL-2 and to express or upregulate some cytokine receptors and other activation molecules and to proliferate. Some of these activated helper T-cells may act directly or indirectly, i.e. assisting effector cytotoxic T-cells or B cells, to destroy cells or tissues expressing the selected antigen. After the termination of the immune response some of the mature clonally selected cells remain as memory helper and memory cytotoxic T-cells, which circulate in the body and rapidly recognize the antigen if appearing again. If the antigen triggering this response is an innocuous environmental antigen the result is allergy, if the antigen is not a foreign antigen, but a self antigen, it can result is autoimmune disease; if the antigen is an antigen from a transplanted organ, the result can be graft rejection.

The immune system has developed to recognize self from non-self. This property enables an organism to survive in an environment exposed to the daily challenges of pathogens. This specificity for non-self and tolerance towards self arises during the development of the T cell repertoire in the thymus through processes of positive and negative selection, which also comprise the recognition and elimination of autoreactive T cells. This type of tolerance is referred to as central tolerance. However, some of these autoreactive cells escape this selective mechanism and pose a potential hazard for the development of autoimmune diseases. To control the autoreactive T cells that have escaped to the periphery, the immune system has peripheral regulatory mechanisms that provide protection against autoimmunity. These mechanisms are a basis for peripheral tolerance.

Cell surface antigens recognized by specific mAbs are generally designated by a CD (Cluster of Differentiation) number assigned by successive International Leukocyte Typing workshops and the term CD45 applied herein refers to the cell surface leukocyte common antigen CD45; and an mAb to that antigen is designated herein as “anti-CD45”.

The leukocyte common antigen (LCA) or CD45 is the major component of anti-lymphocyte globulin (ALG). CD45 belongs to the family of transmembrane tyrosine phosphatases and is both a positive and negative regulator of cell activation, depending upon receptor interaction. The phosphatase activity of CD45 appears to be required for activation of Src-family kinases associated with antigen receptor of B and T lymphocytes (Trowbridge I S et al, Annu Rev Immunol. 1994; 12:85-116). Thus, in T cell activation, CD45 is essential for signal 1 and CD45-deficicient cells have profound defects in TCR-mediated activation events.

The CD45 antigen exists in different isoforms comprising a family of transmembrane glycoproteins. Distinct isoforms of CD45 differ in their extracellular domain structure which arise from alternative splicing of 3 variable exons coding for part of the CD45 extracellular region (Streuli M F. et al, J. Exp. Med. 1987; 166:1548-1566). The various isoforms of CD45 have different extracellular domains, but have the same transmembrane and cytoplasmic segments having two homologous, highly conserved phosphatase domains of approximately 300 residues. Different isoform combinations are differentially expressed on subpopulations of T and B lymphocytes (Thomas M L. et al, Immunol. Today 1988; 9:320-325). Some monoclonal antibodies recognize an epitope common to all the different isoforms, while other mAbs have a restricted (CD45R) specificity, dependent on which of the alternatively spliced exons (A, B or C) they recognize. For example, monoclonal antibodies recognizing the product of exon A are consequently designated CD45RA, those recognizing the various isoforms containing exon B have been designated CD45RB (Beverley P C L et al, Immunol. Supp. 1988; 1:3-5). Antibodies such as UCHL1 selectively bind to the 180 kDa isoform CD45RO (without any of the variable exons A, B or C) which appears to be restricted to a subset of activated T cells, memory cells and cortical thymocytes and is not detected on B cells (Terry L A et al, Immunol. 1988; 64:331-336).

### DESCRIPTION OF THE FIGURES

FIG. 1 shows that the inhibition of primary MLR by the “candidate mAb” is dose-dependent in the range of 0.001 and 10 μg/ml. “Concentration” is concentration of the “candidate mAb”.

FIG. 2 shows the plasmid map of the expression vector HCMV- G1 HuAb-VHQ comprising the heavy chain having the nucleotide sequence SEQ ID NO:12 (3921-4274) in the complete expression vector nucleotide sequence SEQ ID NO:15.

FIG. 3 shows the plasmid map of the expression vector HCMV-G1 HuAb-VHE comprising the heavy chain having the nucleotide sequence SEQ ID NO:11 (3921-4274) in the complete expression vector nucleotide sequence SEQ ID NO:16.

FIG. 4 shows the plasmid map of the expression vector HCMV-K HuAb-humV1 comprising the light chain having the nucleotide sequence SEQ ID NO:14 (3964-4284) in the complete expression vector nucleotide sequence SEQ ID NO:17.

FIG. 5 shows the plasmid map of the expression vector HCMV-K HuAb-humV2 comprising the light chain having the nucleotide sequence SEQ ID NO:13 (3926-4246) in the complete expression vector nucleotide sequence SEQ ID NO:18.

### DESCRIPTION OF THE INVENTION

We have now found a binding molecule which comprises a polypeptide sequence which binds to CD45RO and CD45RB, hereinafter also designated as a “CD45RO/RB binding molecule”. These binding molecule according to the invention may induce immunosuppression, inhibit primary T cell responses and induce T cell tolerance. Furthermore, the binding molecules of the invention inhibit primary mixed lymphocyte responses (MLR). Cells derived from cultures treated with CD45RO/RB binding molecules preferredly also have impaired proliferative responses in secondary MLR even in the absence of CD45RO/RB binding molecules in the secondary MLR. Such impaired proliferative responses in secondary MLR are an indication of the ability of binding molecules of the invention to induce tolerance. Additionally, in vivo administration of CD45RO/RB binding molecule to severe combined immunodeficiency (SCID) mice undergoing xeno-GVHD following injection with human PBMC may prolong mice survival, compared to control treated mice, even though circulating human T cells may still be detected in CD45RO/RB binding molecule treated mice.

By “CD45RO/RB binding molecule” is meant any molecule capable of binding specifically to the CD45RB and CD45RO isoforms of the CD45 antigen, either alone or associated with other molecules. The binding reaction may be shown by standard methods (qualitative assay) including for example any kind of binding assay such as direct or indirect immunofluorescence together with fluorescence microscopy or cytofluorimetric (FACS) analysis, enzyme-linked immunosorbent assay (ELISA) or radioimmunoassay in which binding of the molecule to cells expressing a particular CD45 isoform can be visualized. In addition, the binding of this molecule may result in the alteration of the function of the cells expressing these isoforms. For example inhibition of primary or secondary mixed lymphocyte response (MLR) may be determined, such as an in vitro assay or a bioassay for determining the inhibition of primary or secondary MLR in the presence and in the absence of a CD45RO/RB binding molecule and determining the differences in primary MLR inhibition.

Alternatively, the in vitro functional modulatory effects can also be determined by measuring the PBMC or T cells or CD4⁺ T cells proliferation, production of cytokines, change in the expression of cell surface molecules e.g. following cell activation in MLR, or following stimulation with specific antigen such as tetanus toxoid or other antigens, or with polyclonal stimulators such as phytohemagglutinin (PHA) or anti-CD3 and anti-CD28 antibodies or phorbol esters and Ca²⁺ ionophores. The cultures are set up in a similar manner as described for MLR except that instead of allogeneic cells as stimulators soluble antigen or polyclonal stimulators such as those mentioned above are used. T cell proliferation is measured preferably as described above by ³H-thymidine incorporation.

Cytokine production is measured preferably by sandwich ELISA where a cytokine capture antibody is coated on the surface of a 96-well plate, the supernatants from the cultures are added and incubated for 1 hr at room temperature and a detecting antibody specific for the particular cytokine is then added, following a second-step antibody conjugated to an enzyme such as Horseradish peroxidase followed by the corresponding substrate and the absorbance is measured in a plate reader. The change in cell surface molecules may be preferably measured by direct or indirect immunofluorescence after staining the target cells with antibodies specific for a particular cell surface molecule. The antibody can be either directly labeled with flourochrome or a fluorescently labeled second step antibody specific for the first antibody can be used, and the cells are analysed with a cytofluorimeter.

The binding molecule of the invention has a binding specificity for both CD45RO and CD45RB (“CD45RB/RO binding molecule”).

Preferably the binding molecule binds to CD45RO isoforms with a dissociation constant (Kd) <20 nM, preferably with a Kd<15 nM or <10 nM, more preferably with a Kd<5 nM. Preferably the binding molecule binds to CD45RB isoforms with a Kd<50 nM, preferably with a Kd<15 nM or <10 nM, more preferably with a Kd<5 nM.

In a further preferred embodiment the binding molecule of the invention binds those CD45 isoforms which

1) Include the A and B epitopes but not the C epitope of the CD45 molecule; and/or

2) include the B epitope but not the A and not the C epitope of the CD45 molecule; and/or

3) do not include any of the A, B or C epitopes of the CD45 molecule.

In yet a further preferred embodiment the binding molecule of the invention does not bind CD45 isoforms which include

1) all of the the A, B and C epitopes of the CD45 molecule; and/or

2) both the B and C epitopes but not the A epitope of the CD45 molecule.

In further preferred embodiments the binding molecule of the invention further

1) recognises memory and in vivo alloactivated T cells; and/or

2) binds to its target on human T cells, such as for example PEER cells; wherein said binding preferably is with a Kd<15 nM, more preferably with a Kd<10 nM, most preferably with a Kd<5 nM; and/or

3) inhibits in vitro alloreactive T cell function, preferably with an IC₅₀ of about 5 nM, more preferably with an IC₅₀ of about 1 nM, most preferably with an IC₅₀ of about 0.5 nM or even 0.1 nM; and/or

4) Induces alloantigen-specific T cell tolerance in vitro; and/or

5) prevents lethal xenogeneic graft versus host disease (GvHD) induced in SCID mice by injection of human PBMC when admistiered in an effective amount.

In a further preferred embodiment the binding molecule of the invention binds to the same epitope as the monoclonal antibody “A6” as described by Aversa et al., Cellular Immunology 158, 314-328 (1994).

Due to the above-described binding properties and biological activities, such binding molecules of the invention are particularly useful in medicine, for therapy and/or prophylaxis. Diseases in which binding molecules of the invention are particularly useful include autoimmune diseases, transplant rejection, psoriasis, inflammatory bowel disease and allergies, as will be further set out below.

We have found that a molecule comprising a polypeptide of SEQ ID NO:1and a polypeptide of SEQ ID NO: 2 is a CD45RO/RB binding molecule. We also have found the hypervariable regions CDR1′, CDR2′ and CDR3′ in a CD45RO/RB binding molecule of SEQ ID NO:1, CDR1′ having the amino acid sequence Arg-Ala-Ser-Gln-Asn-Ile-Gly-Thr-Ser-Ile-Gln (RASQNIGTSIQ), CDR2′ having the amino acid sequence Ser-Ser-Ser-Glu-Ser-Ile-Ser (SSSESIS) and CDR3′ having the amino acid sequence Gln-Gln-Ser-Asn-Thr-Trp-Pro-Phe-Thr (QQSNTWPFT).

We also have found the hypervariable regions CDR1, CDR2 and C DR3 in a CD45RO/RB binding molecule of SEQ ID NO:2, CDR1 having the amino acid sequence Asn-Tyr-Ile-Ile-His (NYIIH), CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) and CDR3 having the amino acid sequence Ser-Gly-Pro-Tyr-Ala-Trp-Phe-Asp-Thr (SGPYAWFDT).

CDRs are 3 specific complementary determining regions which are also called hypervariable regions which essentially determine the antigen binding characteristics. These CDRs are part of the variable region, e.g. of SEQ ID NO:1 or SEQ ID NO: 2, respectively, wherein these CDRs alternate with framework regions (FR's) e.g. constant regions. A SEQ ID NO:1 is part of a light chain, e.g. of SEQ ID NO: 3, and a SEQ ID NO:2 is part of a heavy chain, e.g. of SEQ ID NO: 4, in a chimeric antibody according to the present invention. The CDRs of a heavy chain together with the CDRs of an associated light chain essentially constitute the antigen binding site of a molecule of the present invention. It is known that the contribution made by a light chain variable region to the energetics of binding is small compared to that made by the associated heavy chain variable region and that isolated heavy chain variable regions have an antigen binding activity on their own. Such molecules are commonly referred to as single domain antibodies.

In one aspect the present invention provides a molecule comprising at least one antigen binding site, e.g. a CD45RO/RB binding molecule, comprising in sequence the hypervariable regions CDR1, CDR2 and CDR3, said CDR1 having the amino acid sequence Asn-Tyr-Ile-Ile-His (NYIIH), said CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) and said CDR3 having the amino acid sequence Ser-Gly-Pro-Tyr-Ala-Trp-Phe-Asp-Thr (SGPYAWFDT); e.g. and direct equivalents thereof.

In another aspect the present invention provides a molecule comprising at least one antigen binding site, e.g. a CD45RO/RB binding molecule, comprising

a) a first domain comprising in sequence the hypervariable regions CDR1, CDR2 and CDR3, said CDR1 having the amino acid sequence Asn-Tyr-Ile-Ile-His (NYIIH), said CDR2 having the amino acid sequence Tyr-Phe-Asn-Pro-Tyr-Asn-His-Gly-Thr-Lys-Tyr-Asn-Glu-Lys-Phe -Lys-Gly (YFNPYNHGTKYNEKFKG) and said CDR3 having the amino acid sequence Ser-Gly-Pro-Tyr-Ala-Trp-Phe-Asp-Thr (SGPYAWFDT); and

b) a second domain comprising in sequence the hypervariable regions CDR1′, CDR2′ and CDR3′, CDR1′ having the amino acid sequence Arg-Ala-Ser-Gln-Asn-Ile-Gly-Thr-Ser-Ile-Gln (RASQNIGTSIQ), CDR2′ having the amino acid sequence Ser-Ser-Ser-Glu-Ser-Ile-Ser (SSSESIS) and CDR3′ having the amino acid sequence Gln-Gln-Ser-Asn-Thr-Trp-Pro-Phe-Thr (QQSNTWPFT),

e.g. and direct equivalents thereof.

In a preferred embodiment the first domain comprising in sequence the hypervariable regions CDR1, CDR2 and CDR3 is an immunoglobulin heavy chain, and the second domain comprising in sequence the hypervariable regions CDR1′, CDR2′ and CDR3′ is an immunoglobulin light chain.

In another aspect the present invention provides a molecule, e.g. a CD45RO/RB binding molecule, comprising a polypeptide of SEQ ID NO: 1 and/or a polypeptide of SEQ ID NO: 2, preferably comprising in one domain a polypeptide of SEQ ID NO: 1 and in another domain a polypeptide of SEQ ID NO: 2, e.g. a chimeric monoclonal antibody, and in another aspect A molecule, e.g. a CD45RO/RB binding molecule, comprising a polypeptide of SEQ ID NO: 3 and/or a polypeptide of SEQ ID NO: 4, preferably comprising in one domain a polypeptide of SEQ ID NO: 3 and in another domain a polypeptide of SEQ ID NO: 4, e.g. a chimeric monoclonal antibody.

When the antigen binding site comprises both the first and second domains or a polypeptide of SEQ ID NO:1 or SEQ ID NO:3, respectively, and a polypeptide of SEQ ID NO: 2 or of SEQ ID NO:4, respectively, these may be located on the same polypeptide, or, preferably each domain may be on a different chain, e.g. the first domain being part of an heavy chain, e.g. immunoglobulin heavy chain, or fragment thereof and the second domain being part of a light chain, e.g. an immunoglobulin light chain or fragment thereof.

We have further found that a CD45RO/RB binding molecule according to the present invention is a CD45RO/RB binding molecule in mammalian, e.g. human, body environment. A CD45RO/RB binding molecule according to the present invention can thus be designated as a monoclonal antibody (mAb), wherein the binding activity is determined mainly by the CDR regions as described above, e.g. said CDR regions being associated with other molecules without binding specifity, such as framework, e.g. constant regions, which are substantially of human origin.

In another aspect the present invention provides a CD45RO/RB binding molecule which is not the monoclonal antibody “A6” as described by Aversa et al., Cellular Immunology 158, 314-328 (1994), which is incoporated by reference for the passages characterizing A6.

In another aspect the present invention provides a CD45RO/RB binding molecule according to the present invention which is a chimeric, a humanised or a fully human monoclonal antibody.

Examples of a CD45RO/RB binding molecules include chimeric or humanised antibodies e.g. derived from antibodies as produced by B-cells or hybridomas and or any fragment thereof, e.g. F(ab′)2 and Fab fragments, as well as single chain or single domain antibodies. A single chain antibody consists of the variable regions of antibody heavy and light chains covalently bound by a peptide linker, usually consisting of from 10 to 30 amino acids, preferably from 15 to 25 amino acids. Therefore, such a structure does not include the constant part of the heavy and light chains and it is believed that the small peptide spacer should be less antigenic than a whole constant part. By a chimeric antibody is meant an antibody in which the constant regions of heavy and light chains or both are of human origin while the variable domains of both heavy and light chains are of non-human (e.g. murine) origin. By a humanised antibody is meant an antibody in which the hypervariable regions (CDRs) are of non-human (e.g. murine) origin while all or substantially all the other part, e.g. the constant regions and the highly conserved parts of the variable regions are of human origins. A humanised antibody may however retain a few amino acids of the murine sequence in the parts of the variable regions adjacent to the hypervariable regions.

Hypervariable regions, i.e. CDR's according to the present invention may be associated with any kind of framework regions, e.g. constant parts of the light and heavy chains, of human origin. Suitable framework regions are e.g. described in “Sequences of proteins of immunological interest”, Kabat, E. A. et al, US department of health and human services, Public health service, National Institute of health. Preferably the constant part of a human heavy chain may be of the IgG1 type, including subtypes, preferably the constant part of a human light chain may be of the κ or λ type, more preferably of the κ type. A preferred constant part of a heavy chain is a polypeptide of SEQ ID NO: 4 (without the CDR1′, CDR2′ and CDR3′ sequence parts which are specified above) and a preferred constant part of a light chain is a polypeptide of SEQ ID NO: 3 (without the CDR1, CDR2 and CDR3 sequence parts which are specified above).

We also have found a humanised antibody comprising a light chain variable region of amino acid SEQ ID NO:7 or of amino acid SEQ ID NO:8, which comprises CDR1′, CDR2′ and CDR3′ according to the present invention and a heavy chain variable region of SEQ:ID NO:9 or of SEQ:ID NO:10, which comprises CDR1, CDR2 and CDR3 according to the present invention.

In another aspect the present invention provides a humanised antibody comprising a polypeptide of SEQ ID NO:9 or of SEQ ID NO:10 and a polypeptide of SEQ ID NO:7 or of SEQ ID NO:8.

In another aspect the present invention provides a humanised antibody comprising

a polypeptide of SEQ ID NO:9 and a polypeptide of SEQ ID NO:7,

a polypeptide of SEQ ID NO:9 and a polypeptide of SEQ ID NO:8,

a polypeptide of SEQ ID NO:10 and a polypeptide of SEQ ID NO:7, or

a polypeptide of SEQ ID NO:10 and a polypeptide of SEQ ID NO:8.

A polypeptide according to the present invention, e.g. of a herein specified sequence, e.g. of CDR1, CDR2, CDR3, CDR1′, CDR2′, CDR3′, or of a SEQ ID NO:1, SEQ ID NO:2, SEQ ID NO:3, SEQ ID NO:4, SEQ ID NO:7, SEQ ID NO:8, SEQ ID NO:9 or SEQ ID NO:10 includes direct equivalents of said (poly)peptide (sequence); e.g. including a functional derivative of said polypeptide. Said functional derivative may include covalent modifications of a specified sequence, and/or said functional derivative may include amino acid sequence variants of a specified sequence.

“Polypeptide”, if not otherwise specified herein, includes any peptide or protein comprising amino acids joined to each other by peptide bonds, having an amino acid sequence starting at the N-terminal extremity and ending at the C-terminal extremity. Preferably the polypeptide of the present invention is a monoclonal antibody, more preferred is a chimeric (V-grafted) or humanised (CDR-grafted) monoclonal antibody. The humanised (CDR-grafted) monoclonal antibody may or may not include further mutations introduced into the framework (FR) sequences of the acceptor antibody.

A functional derivative of a polypeptide as used herein includes a molecule having a qualitative biological activity in common with a polypeptide to the present invention, i.e. having the ability to bind to CD45RO and CD45RB. A functional derivative includes fragments and peptide analogs of a polpypeptide according to the present invention. Fragments comprise regions within the sequence of a polypeptide according to the present invention, e.g. of a specified sequence. The term “derivative” is used to define amino acid sequence variants, and covalent modifications of a polypeptide according to the present invention. e.g. of a specified sequence. The functional derivatives of a polypeptide according to the present invention, e.g. of a specified sequence, preferably have at least about 65%, more preferably at least about 75%, even more preferably at least about 85%, most preferably at least about 95% overall sequence homology with the amino acid sequence of a polypeptide according to the present invention, e.g. of a specified sequence, and substantially retain the ability to bind to CD45RO and CD45RB.

The term “covalent modification” includes modifications of a polypeptide according to the present invention, e.g. of a specified sequence; or a fragment thereof with an organic proteinaceous or non-proteinaceous derivatizing agent, fusions to heterologous polypeptide sequences, and post-translational modifications. Covalent modified polypeptides, e.g. of a specified sequence, still have the ability bind to CD45RO and CD45RB by crosslinking. Covalent modifications are traditionally introduced by reacting targeted amino acid residues with an organic derivating agent that is capable of reacting with selected sides or terminal residues, or by harnessing mechanisms of post-translational modifications that function in selected recombinant host cells. Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deaminated under mildly acidic conditions. Other post-translational modifications include hydroxylation of proline and lysine, phosphorylation of hydroxyl groups of seryl, tyrosine or threonyl residues, methylation of the α-amino groups of lysine, arginine, and histidine side chains, see e.g. T. E. Creighton, Proteins: Structure and Molecular Properties, W. H. Freeman & Co., San Francisco, pp. 79-86 (1983). Covalent modifications e.g. include fusion proteins comprising a polypeptide according to the present invention, e.g. of a specified sequence and their amino acid sequence variants, such as immunoadhesins, and N-terminal fusions to heterologous signal sequences.

“Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- or C-terminal extensions nor insertions shall be construed as reducing identity or homology. Methods and computer programs for the alignment are well known.

“Amino acid(s)” refer to all naturally occurring L-α-amino acids, e.g. and including D-amino acids. The amino acids are identified by either the well known single-letter or three-letter designations.

The term “amino acid sequence variant” refers to molecules with some differences in their amino acid sequences as compared to a polypeptide according to the present invention, e.g. of a specified sequence. Amino acid sequence variants of a polypeptide according to the present invention, e.g. of a specified sequence, still have the ability to bind to CD45RO and CD45RB. Substitutional variants are those that have at least one amino acid residue removed and a different amino acid inserted in its place at the same position in a polypeptide according to the present invention, e.g. of a specified sequence. These substitutions may be single, where only one amino acid in the molecule has been substituted, or they may be multiple, where two or more amino acids have been substituted in the same molecule. Insertional variants are those with one or more amino acids inserted immediately adjacent to an amino acid at a particular position in a polypeptide according to the present invention, e.g. of a specified sequence. Immediately adjacent to an amino acid means connected to either the α-carboxy or α-amino functional group of the amino acid. Deletional variants are those with one or more amino acids in a polypeptide according to the present invention, e.g. of a specified sequence, removed. Ordinarily, deletional variants will have one or two amino acids deleted in a particular region of the molecule.

We also have found the polynucleotide sequences of

GGCCAGTCAGAACATTGGCACAAGCATACAGTG, encoding the amino acid sequence of CDR1,

TTCTTCTGAGTCTATCTCTGG; encoding the amino acid sequence of CDR 2,

ACAAAGTAATACCTGGCCATTCACGTT encoding the amino acid sequence of CDR 3,

TTATATTATCCACTG, encoding the amino acid sequence of CDR1′,

TTTTAATCCTTACAATCATGGTACTAAGTACAATGAGAAGTTCAAAGGCAG encoding the amino acid sequence of CDR2,

AGGACCCTATGCCTGGTTTGACACCTG encoding the amino acid sequence of CDR3′,

SEQ ID NO:5 encoding a polypeptide of SEQ ID NO: 1, i.e. the variable region of a light chain of an mAb according to the present invention;

SEQ ID NO:6 encoding a polypeptide of SEQ ID NO:2, i.e. the variable region of the heavy chain of an mAb according to the present invention;

SEQ ID NO:11 encoding a polypeptide of SEQ ID NO:9. i.e. a heavy chain variable region including CDR1, CDR2 and CDR3 according to the present invention;

SEQ ID NO:12 encoding a polypeptide of SEQ ID NO:10, i.e. a heavy chain variable region including CDR1, CDR2 and CDR3 according to the present invention;

SEQ ID NO:13 encoding a polypeptide of SEQ ID NO:7, i.e. a light chain variable region including CDR1′, CDR2′ and CDR3′ according to the present invention; and

SEQ ID NO:14 encoding a polypeptide of SEQ ID NO:8, i.e. a light chain variable region including CDR1′, CDR2′ and CDR3′ according to the present invention.

In another aspect the present invention provides isolated polynucleotides comprising

polynucleotides encoding a CD45RO/RB binding molecule, e.g. encoding the amino acid sequence of CDR1, CDR2 and CDR3 according to the present invention and/or, preferably and, polynucletides encoding the amino acid sequence of CDR1′, CDR2′ and CDR3′ according to the present invention; and

Polynucleotides comprising a polynucleotide of SEQ ID NO: 5 and/or, preferably and, a polynucleotide of SEQ ID NO: 6; and

Polynucleotides comprising polynucleotides encoding a polypeptide of SEQ ID NO:7 or SEQ ID NO:8 and a polypeptide of SEQ ID NO:9 or SEQ ID NO:10; e.g. encoding

a polypeptide of SEQ ID NO:7 and a polypeptide of SEQ ID NO:9,

a polypeptide of SEQ ID NO:7 and a polypeptide of SEQ ID NO:10,

a polypeptide of SEQ ID NO:8 and a polypeptide of SEQ ID NO:9, or

a polypeptide of SEQ ID NO:8 and a polypeptide of SEQ ID NO:10; and

Polynucleotides comprising a polynucleotide of SEQ ID NO:11 or of SEQ ID NO:12 and a polynucleotide of SEQ ID NO:13 or a polynucleotide of SEQ ID NO:14, preferably comprising

a polynucleotide of SEQ ID NO:11 and a polynucleotide of SEQ ID NO:13,

a polynucleotide of SEQ ID NO:11 and a polynucleotide of SEQ ID NO:14,

a polynucleotide of SEQ ID NO:12 and a polynucleotide of SEQ ID NO:13, or

a polynucleotide of SEQ ID NO:12 and a polynucleotide of SEQ ID NO:14.

“Polynucleotide”, if not otherwise specified herein, includes any polyribonucleotide or polydeoxyribonucleotide, which may be unmodified RNA or DNA, or modified RNA or DNA, including without limitation single and double stranded RNA, and RNA that is a mixture of single- and double-stranded regions.

A polynucleotide according to the present invention, e.g. a polynucleotide encoding the amino acid sequence CDR1, CDR2, CDR3, CDR¹′, CDR2′, CDR3′, or of SEQ ID NO:1, SEQ ID NO:2, SEQ ID NO:3, SEQ ID NO:4, SEQ ID NO:7, SEQ ID NO:8, SEQ ID NO:9 or SEQ ID NO:10, respectively, such as a polynucleotide of SEQ ID NO:5, SEQ ID NO:6, SEQ ID NO:11, SEQ ID NO:12, SEQ ID NO:13 or SEQ ID NO:14, respectively, includes allelic variants thereof and/or their complements; e.g. including a polynucleotide that hybridizes to the nucleotide sequence of SEQ ID NO: 5, SEQ ID NO:6, SEQ ID NO:11, SEQ ID NO:12, SEQ ID NO:13 or SEQ ID NO:14, respectively; e.g. encoding a polypeptide having at least 80% identity to SEQ ID NO:1, SEQ ID NO:2, SEQ ID NO:3, SEQ ID NO:4, SEQ ID NO:7, SEQ ID NO:8, SEQ ID NO:9 or SEQ ID NO:10, respectively, e.g. including a functional derivative of said polypeptide, e.g. said functional derivative having at least 65% homology with SEQ ID NO:1, SEQ ID NO:2, SEQ ID NO:3, SEQ ID NO:4, SEQ ID NO:7, SEQ ID NO:8, SEQ ID NO:9 or SEQ ID NO:10, respectively, e.g. said functional derivative including covalent modifications of SEQ ID NO:1, SEQ ID NO:2, SEQ ID NO:3, SEQ ID NO:4, SEQ ID NO:7, SEQ ID NO:8, SEQ ID NO:9 or SEQ ID NO:10, respectively, e.g. said functional derivative including amino acid sequence variants of SEQ ID NO:1, SEQ ID NO:2, SEQ ID NO:3, SEQ ID NO:4, SEQ ID NO:7, SEQ ID NO:8, SEQ ID NO:9 or SEQ ID NO:10, respectively; e.g. a SEQ ID NO:5, SEQ ID NO:6, SEQ ID NO:11, SEQ ID NO:12, SEQ ID NO:13 or SEQ ID NO:14, respectively includes a sequence, which as a result of the redundancy (degeneracy) of the genetic code, also encodes a polypeptide of SEQ ID NO: 1, SEQ ID NO:2, SEQ ID NO:3, SEQ ID NO:4, SEQ ID NO:7, SEQ ID NO:8, SEQ ID NO:9 or SEQ ID NO:10, respectively, or encodes a polypeptide with an amino acid sequence which has at least 80% identity with the amino acid sequence of SEQ ID NO:1, SEQ ID NO:2, SEQ ID NO:3, SEQ ID NO:4, SEQ ID NO:7, SEQ ID NO:8, SEQ ID NO:9 or SEQ ID NO:10, respectively.

A CD45RO/RB binding molecule, e.g. which is a chimeric or humanised antibody, may be produced by recombinant DNA techniques. Thus, one or more DNA molecules encoding the CD45RO/RB may be constructed, placed under appropriate control sequences and transferred into a suitable host (organism) for expression by an appropriate vector.

In another aspect the present invention provides a polynucleotide which encodes a single, heavy and/or a light chain of a CD45RO/RB binding molecule according to the present invention; and the us of a polynucleotide according to the present invention for the production of a CD45RO/RB binding molecule according to the present invention by recombinant means.

A CD45RO/RB binding molecule may be obtained according, e.g. analogously, to a method as conventional together with the information provided herein, e.g. with the knowledge of the amino acid sequence of the hypervariable or variable regions and the polynucleotide sequences encoding these regions. A method for constructing a variable domain gene is e.g. described in EP 239 400 and may be briefly summarized as follows: A gene encoding a variable region of a mAb of whatever specificity may be cloned. The DNA segments encoding the framework and hypervariable regions are determined and the DNA segments encoding the hypervariable regions are removed. Double stranded synthetic CDR cassettes are prepared by DNA synthesis according to the CDR and CDR′ sequences as specified herein. These cassettes are provided with sticky ends so that they can be ligated at junctions of a desired framework of human origin. Polynucleotides encoding single chain antibodies may also be prepared according to, e.g. analogously, to a method as conventional. A polynucleotide according to the present invention thus prepared may be conveniently transferred into an appropriate expression vector.

Appropriate cell lines may be found according, e.g. analogously, to a method as conventional. Expression vectors, e.g. comprising suitable promotor(s) and genes encoding heavy and light chain constant parts are known e.g. and are commercially available. Appropriate hosts are known or may be found according, e.g. analogously, to a method as conventional and include cell culture or transgenic animals.

In another aspect the present invention provides an expression vector comprising a polynucleotide encoding a CD45RO/RB binding molecule according to the present invention, e.g. of sequence SEQ ID NO:15, SEQ ID NO:16, SEQ ID NO:17 or SEQ ID NO:18.

In another aspect the present invention provides

An expression system comprising a polynucleotide according to the present invention wherein said expression system or part thereof is capable of producing a CD45RO/RB binding molecule according to the present invention, when said expression system or part thereof is present in a compatible host cell; and

An isolated host cell which comprises an expression system as defined above.

We have further found that a CD45RO/RB binding molecule according to the present invention inhibit primary alloimmune responses in a dose-dependent fashion as determined by in vitro MLR. The results indicate that the cells which had been alloactivated in the presence of a CD45RO/RB binding molecule according to the present invention are impaired in their responses to alloantigen. This confirms the indication that a CD45RO/RB binding molecule according to the present invention can act directly on the effector alloreactive T cells and modulate their function. In addition, the functional properties of T cells derived from the primary MLR were further studied in restimulation experiments in secondary MLR, using specific stimulator cells or third-party stimulators to assess the specificity of the observed functional effects. We have found that the cells derived from primary MLRs in which a CD45RO/RB binding molecule according to the present invention is present, were impaired in their ability to respond to subsequent optimal stimulation with specific stimulator cells, although there was no antibody added to the secondary cultures. The specificity of the inhibition was demonstrated by the ability of cells treated with a CD45RO/RB binding molecule according to the present invention to respond normally to stimulator cells from unrelated third-party donors. Restimulation experiments using T cells derived from primary MLR cultures thus indicate that the cells which had been alloactivated a CD45RO/RB binding molecule according to the present invention are hyporesponsive, i.e. tolerant, to the original alloantigen. Further biological activities are described in example 7.

Furthermore we have found that cell proliferation in cells pre-treated with a CD45RO/RB binding molecule according to the present invention could be rescued by exogenous IL-2. This indicates that treatment of alloreactive T cells with a CD45RO/RB binding molecule according to the present invention includes a state of tolerance. Indeed, the reduced proliferative responses observed in cells treated with a CD45RO/RB binding molecule according to the present invention, was due to impairement of T cell function, and these cells were able to respond to exogenous IL-2, indicating that these cells are in an anergic, true unresponsive state. The specificity of this response was shown by the ability of cells treated with a CD45RO/RB binding molecule according to the present invention to proliferate normally to unrelated donor cells to the level of the control treated cells.

In addition experiments indicate that the binding of a CD45RO/RB binding molecule according to the present invention to CD45RO and CD45RB may inhibit the memory responses of peripheral blood mononuclear cells (PBMC) from immunized donors to specific recall antigen. Binding of a CD45RO/RB binding molecule according to the present invention to CD45RO and CD45RB thus is also effective in inhibiting memory responses to soluble Ag. The ability of a CD45RO/RB binding molecule according to the present invention to inhibit recall responses to tetanus in PBMC from immunized donors indicate that the a CD45RO/RB binding molecule according to the present invention is able to target and modulate the activation of memory T cells. E.g. these data indicate that a CD45RO/RB binding molecule according to the present invention in addition to recognizing alloreactive and activated T cells is able to modulate their function, resulting in induction of T cell anergy. This property may be important in treatment of ongoing immune responses to autoantigens and allergens and possibly to alloantigens as seen in autoimmune diseases, allergy and chronic rejection, and diseases, such as psoriasis, inflammatory bowel disease, where memory responses play a role in the maintenance of disease state. It is believed to be an important feature in a disease situation, such as in autoimmune diseases in which memory responses to autoantigens may play a major role for the disease maintenance.

We have also found that a CD45RO/RB binding molecule according to the present invention may modulate T cell proliferative responses in a mixed lymphocyte response (MLR) in vivo, i.e. a CD45RO/RB binding molecule according to the present invention was found to have corresponding inhibitory properties in vivo testing.

A CD45RO/RB binding molecule according to the present invention may thus have immunosuppressive and tolerogenic properties and may be useful for in vivo and ex-vivo tolerance induction to alloantigens, autoantigens, allergens and bacterial flora antigens, e.g. a CD45RO/RB binding molecule according to the present invention may be useful in the treatment and prophylaxis of diseases e.g. including autoimmune diseases, such as, but not limited to, rheumatoid arthritis, autoimmune thyroditis, Graves disease, type I and type II diabetes, multiple sclerosis, systemic lupus erythematosus, Sjögren syndrome, sclerodemra, autoimmune gastritis, glomerulonephritis, transplant rejection, e.g. organ and tissue allograft and xenograft rejection, graft versus host disease (GVHD), and also psoriasis, inflammatory bowel disease and allergies.

In another aspect the present invention provides the use of a CD45RO/RB binding molecule according to the present invention as a pharmaceutical, e.g. in the treatment and prophylaxis of autoimmune diseases, transplant rejection, psoriasis, inflammatory bowel disease and allergies.

In another aspect the present invention provides a CD45RO/RB binding molecule according to the present invention for the production of a medicament in the treatment and prophylaxis of diseases associated with autoimmune diseases, transplant rejection, psoriasis, inflammatory bowel disease and allergies.

In another aspect the present invention provides a pharmaceutical composition comprising a CD45RO/RB binding molecule according to the present invention in association with at least one pharmaceutically acceptable carrier or diluent.

A pharmaceutical composition may comprise further, e.g. active, ingredients, e.g. other immunomodulatory antibodies such as, but not confined to anti-ICOS, anti-CD154, anti-CD134L or recombinant proteins such as, but not confined to rCTLA-4 (CD152), rOX40 (CD134), or immunomodulatory corn pounds such as, but not confined to cyclosporin A, FTY720, RAD, rapamycin, FK506, 15-deoxyspergualin, steroids.

In another aspect the present invention provides a method of treatment and/or prophylaxis of diseases associated with autoimmune diseases, transplant rejection, psoriasis, inflammatory bowel disease and allergies comprising administering to a subject in need of such treatment and/or prophylaxis an effective amount of a CD45RO/RB binding molecule according to the present invention, e.g. in the form of a pharmaceutical composition according to the present invention.

Autoimune diseases to be treated with binding molecule of the present invention further include, but are not limited to, rheumatoid arthritis, autoimmune thyroditis, Graves disease, type I and type II diabetes, multiple sclerosis, systemic lupus erythematosus, Sjögren syndrome, scleroderma, autoimmune gastritis, glomerulonephritis; transplant rejection, e.g. organ and tissue allograft and xenograft rejection and graft-versus-host disease (GVHD).

### EXAMPLES

The invention will be more fully understood by reference to the following examples. They should not, however, be construed as limiting the scope of the invention. In the following examples all temperatures are in degree Celsius.

The “candidate mAb′ ” or “chimeric antibody” is a CD45RO/RB binding molecule according to the present invention comprising light chain of SEQ ID NO:3 and heavy chain of SEQ ID NO:4.

The following abbreviations are used:

| | |
|---|---|
| ELISA | enzyme linked immuno-sorbant assay |
| FACS | fluorescence activated cell sorting |
| FITC | fluorescein isothiocyanate |
| FBS | foetal bovine serum |
| GVHD | graft-vs-host disease |
| HCMV | human cytomegalovirus promoter |
| IgE | immunoglobulin isotype E |
| IgG | immunoglobulin isotype G |
| PBS | phosphate-buffered saline |
| PCR | polymerase chain reaction |
| xGVHD | xeno-graft-vs-host disease |

### Example 1

### Primary Mixed Lymphocyte Response (MLR)

Cells

Blood samples are obtained from healthy human donors. Peripheral blood mononuclear cells (PBMC) are isolated by centrifugation over Ficoll-Hypaque (Pharmacia LKB) from leukocytes from whole peripheral blood, leukopheresis or buffy coats with known blood type, but unknown HLA type. In some MLR experiments, PBMC are directly used as the stimulator cells after the irradiation at 40 Gy. In the other experiments, T cells were depleted from PBMC by using CD2 or CD3 Dynabeads (Dynal, Oslo, Norway). Beads and contaminating cells are removed by magnetic field. T cell-depleted PBMC are used as simulator cells after the irradiation.

PBMC, CD3⁺ T cells or CD4⁺ T cells are used as the responder cells in MLR. Cells are prepared from different donors to stimulator cells. CD3⁺ T cells are purified by negative selection using anti-CD16 mAb (Zymed, Calif.), goat anti-mouse IgG Dynabeads, anti-CD14 Dynabeads, CD19 Dynabeads. In addition anti-CD8 Dynabeads are used to purify CD4⁺ T cells. The cells obtained are analyzed by FACScan or FACSCalibur (Becton Dickinson & Co., CA) and the purity of the cells obtained was >75%. Cells are suspended in RPMI1640 medium, supplemented with 10 % heat-inactivated FBS, penicillin, streptomycin and L-glutamine.

Reagents

The chimeric anti-CD45RO/RB mAb “candidate mAb” and an isotype matched control chimeric antibody is also generated. Mouse (Human) control IgG₁ antibody specific for KLH (keyhole limpet hemocyanin) or recombinant human IL-10 is purchased from BD Pharmingen (San Diego, Calif.). Anti-human CD154 mAb 5c8 is according to Lederman et al 1992.

Primary Mixed Lymphocyte Response (MLR)

Aliquots of 1×10⁵ PBMC or 5×10⁴ of CD3⁺ or CD4⁺ cells are mixed with 1×10⁵ irradiated PBMC or 5×10⁴ T cells-depleted irradiated (50 Gy) PBMC in the each well of 96-well culture plates (Costar, Cambridge, Mass.) in the presence of the indicated mAb or absence of Ab. In some experiments, F(ab′)₂ fragment of goat anti-mouse Ig or goat antihuman Ig specific for Fc portion (Jackson ImmunoResearch, West Grove, Pa.) is added at 10 μg/ml in addition to the candidate mAb To ensure optimal in vitro cross-linking of the target CD45 molecules. The mixed cells are cultured for 4 or 5 days at 37° C. In 5% CO₂ and proliferation is determined by pulsing the cells with ³H-thymidine for the last 16-20 hours of culture. Other experiments are similar to those described above, but with the following exceptions: 1) Medium used is EX-VIVO (Bio-Whittaker) containing 10% FBS and 1% human plasma; 2) Anti-mouse total IgG (5 μg/ml) is used as secondary cross-linking step; 3) irradiation of stimulator cells is 60 Gy.

Primary MLR is performed in the presence of the “candidate mAb” or control chimeric IgG₁ (10 μg/ml) both with a second step reagent, F(ab′)₂ fragment of goat anti-human Ig specific for Fc portion (10 μg/ml). Percentage inhibition by the “candidate mAb” is calculated in comparison with the cell proliferation in the presence of control IgG₁. Results are shown in TABLE 1 below:

TABLE 1
Inhibition of primary MLR by 10 μg/ml of a candidate mAb
according to the present invention

| Responder | Stimulator (Irr. PBMC) | % of Inhibition |
|---|---|---|
| #211 CD4 | #219 CD3 | 63.51 |
| #220 CD4 | #219 CD3 depl. | 63.07 |
| #227 CD4 | #220 CD3 depl. | 65.96 |
| #229 CD4 | #219 CD3 depl. | 50.76 |

| | |
|---|---|
| Average ± SD | 60.83 ± 6.83* |

*Significantly different from control value (P < 0.001)

A candidate mAb according to the present invention inhibits primary MLR as can be seen from TABLE 1. The average inhibitory effect is 60.83±6.83 % in four different donors-derived CD4⁺ T cells and statistically significant.

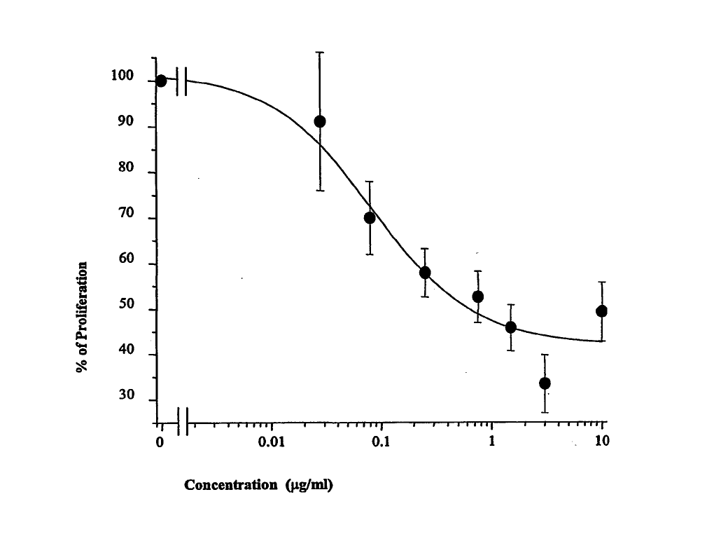

The inhibition of primary MLR by the “candidate mAb” is shown to be dose-dependent in the range of 0.001 and 10 μg/ml of the “candidate mAb” as shown in FIG. 1. The IC₅₀ for the inhibition of primary MLR by a “candidate mAb” is determined from the results of three separate MLR experiments using one donor PBMC as responder cells. Thus, responder CD4⁺ T cells from Donor #229 and #219 and irradiated PBMC depleted of T cells as stimulators are mixed in the presence of a “candidate mAb” or control chimeric Ab with 10 μg/ml of F(ab′)₂ fragment of goat anti-human Ig. Experiments are repeated 3 times and percentage of proliferation in the presence of a “candidate mAb” is calculated in comparison with the T cell proliferation in the presence of control Ab. IC₅₀ value is determined using Origin (V. 6.0™). The cellular activity IC₅₀ value is calculated to be 0.87±0.35 nM (0.13±0.052 μg/ml).

### Example 2

### Secondary MLR

In order to assess whether a “candidate mAb” induces unresponsiveness of CD4⁺ T cells to specific alloantigens, secondary MLR is performed in the absence of any antibodies after the primary MLC. CD4⁺ T cells are cultured with irradiated allogeneic stimulator cells (T cells-depleted PBMC) in the presence of the indicated antibody in 96-well culture plates for 10 days (primary MLC). Then, cells are collected, layered on a Ficoll-Hypaque gradient to remove dead cells, washed twice with RPMI, and restimulated with the same stimulator, 3^{rd} party stimulator cells or IL-2 (50 U/ml). The cells are cultured for 3 days and the proliferative response is determined by pulsing the cells with ³H-thymidine for the last 16-20 hours of culture.

Specifically, CD4⁺ T cells are cultured with irradiated allogeneic stimulator cells (T cells-depleted PBMC taken from other donors) in the presence of 10 μg/ml of the “candidate mAb”, control IgG₁ chimeric Ab and F(ab′)₂ fragment of goat anti-human Ig. Primary MLR proliferation is determined on day 5. For secondary MLR, the responder and stimulator cells are cultured for 10 days in the presence of the “candidate mAb”, then the cells are harvested, washed twice in RPMI1640 and restimulated with specific stimulator, third-party stimulators or IL-2 (50 U/ml) in the absence of any Ab. Cell proliferation is determined on day 3. Results set out in TABLE 2:

TABLE 2

| Responder CD4+ T cells Donor # | % Inhibition of 2^{ry} MLR |
|---|---|
| #211 | 49.90* |
| #220 | 59.33* |
| #227 | 58.68* |

*Significantly different from control value (p = < 0.001 determined by t-test, SigmaStat V.2.03).
#p = < 0.046

In order to test whether the impaired proliferation is due to unresponsivess as a consequence of the treatment with a “candidate mAb”, the cells derived from primary MLR are cultured in the presence of IL-2 (50 U/ml). Addition of IL-2 results in the rescue of proliferative responses of the T cells which had been treated with a “candidate mAb” in primary MLR, to levels similar to those observed in the presence of IgG₁ control Ab. These data indicate that the impaired secondary response in T cells treated with a “candidate mAb” is due to to functional alteration of the responder T cells which become unresponsive to the specific stimulator cells.

Percentage inhibition is calculated according to the following formula:

$$\frac{c . p . m . with control A b - c . p . m . with “ candidate mAb}{c . p . m . with control A b} \times 100$$

Statistical analysis is performed using SigmaStat (Vers. 2.03).

The data is analyzed by two-way ANOVA followed by Dunnett method. In all test procedures probabilities <0.05 are considered as significant. In some experiments t-test is used (SigmaStat V.2.03).

### Example 3

### In Vivo Survival Studies in SCID-mice

Engraftment of hu-PBL in SCID Mice

Human peripheral blood mononuclear cells (PBMC) are injected intraperitoneally into SCID mice C.B 17 /GbmsTac-Prkdc^{scid} Lyst^{bg} mice (Taconic, Germantown, N.Y.) in an amount sufficient to induce a lethal xenogeneic graft-versus-host disease (xGvHD) in >90% of the mice within 4 weeks after cell transfer. Such treated SCID mice are hereinafter designated as hu-PBL-SCID mice

Mab-treatment of hu-PBL-SCID Mice

Hu-PBL-SCID mice are treated with a “candidate mAb” or mouse or chimeric isotype matched mAb controls at day 0, immediately after PBMC injection, at day 3, day 7 and at weekly intervals thereafter. Mabs are delivered subcutaneously in 100 μl PBS at a final concentration of 5 mg/kg body weight. The treatment was stopped when all control mice were dead.

Evaluation of Treatment Results

The main criterion to assess the efficacy of a “candidate mAb” in this study was the survival of the hu-PBL-SCID mice. The significance of the results is evaluated by the statistical method of survival analysis using the Log-rank test (Mantel method) with the help of the Systat v9.01 software. The method of survival analysis is a non-parametric test, which not only consider whether a particular mouse is still alive but also whether if it was sacrificed for reasons irrelevant to the treatment/disease such as the requirement of perform in vitro analysis with its organs/cells. Biopsies of liver, lung, kidney and spleen are obtained from dead mice for further evaluation. In addition, hu-PBL-SCID mice are weighed at the beginning (before cell transfer) and throughout (every two days) the experiment as an indirect estimation of their health status. Linear regression lines were generated using the body weight versus days post-PBMC transfer values obtained from each mouse and subsequently, their slopes (control versus anti-CD45 treated mice) were compared using the non-parametric Mann-Whitney test.

Results

All hu-PBL-SCID mice treated with mouse mAb controls had infiltrated human leukocytes in the lung, liver and spleen and died (4/4) within ca. 2 to 3 weeks after cell transfer. Death is a likely consequence of xGvHD. Control mAb-treated mice furthermore lost weight in a linear manner, ca. 10% and more within 3 weeks.

All hu-PBL-SCID mice treated with a “candidate mAb” survived (4/4) without any apparent sign of disease more than 4 weeks, even although “candidate mAb”-treatment was stopped after 3 weeks. “Candidate mAb”-treated mice increased weight in a linear manner, up to ca. 5% within 4 weeks.

### Example 4

### Expression of Antibodies of the Invention

Expression of Humanised Antibody Comprising a SEQ ID NO:7, SEQ ID NO:8, SEQ ID NO:9, or SEQ ID NO:10

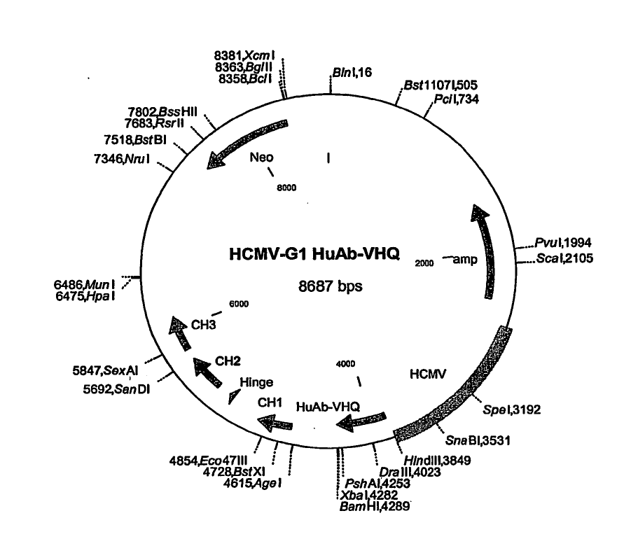

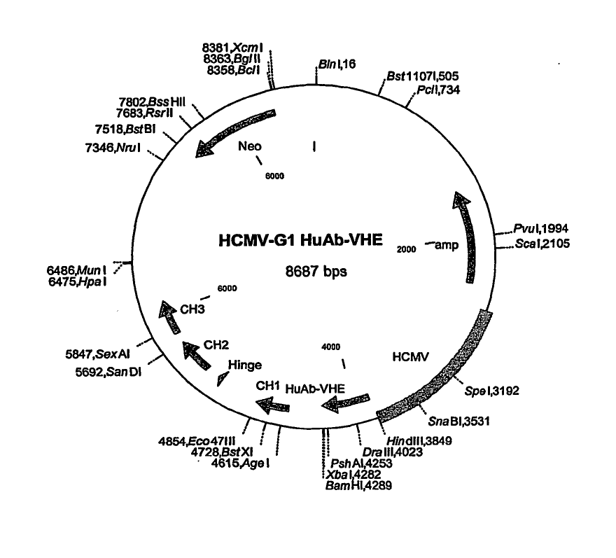

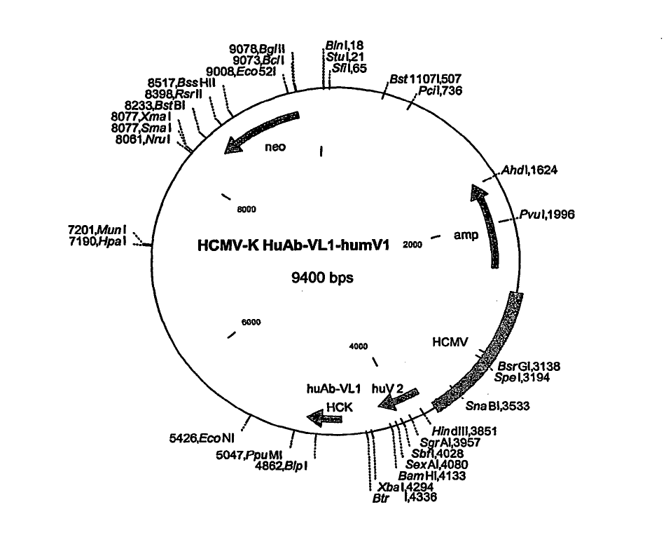

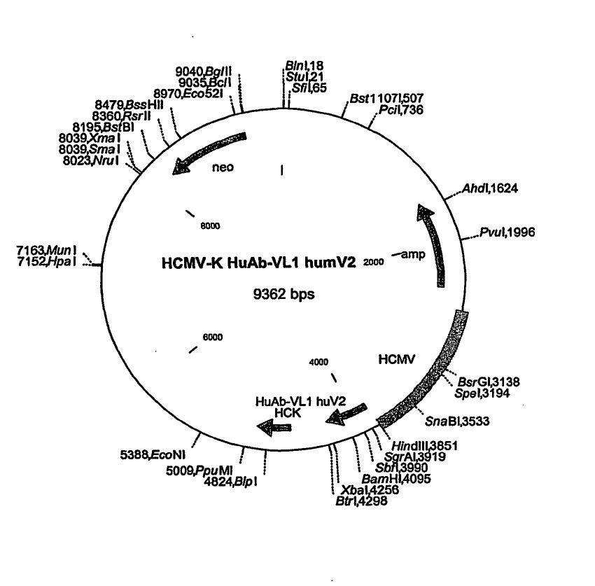

Expression vectors according to the plasmid map shown in FIGS. 2 to 5 are constructed, comprising the corresponding nucleotides encoding the amino acid sequence of humanised light chain variable region humV1 (SEQ ID NO:7), humanised light chain variable region humV2 (SEQ ID NO:8), humanised heavy chain variable region VHE (SEQ ID NO:9), or humanised heavy chain variable region VHQ (SEQ ID NO:10), respectively. These expression vectors have the DNA (nucleotide) sequences SEQ ID NO 15, SEQ ID NO 16, SEQ ID NO 17, or SEQ ID NO 18, respectively.

Construction of Humanised Antibody Heavy and Light Chain Expression Vectors

Human Kappa Light Chain Expression Vectors for Versions VLh and VLm

In order to construct the final expression vector encoding for the complete humanised light chain of human kappa isotype, DNA fragments encoding the complete light chain variable regions (VLh and VLm) were excised from the VLh and VLm containing PCR-Script cloning vectors (Stratagene) (VLm region) using HindIII and BgIII. The gel-purified fragments were then subcloned into the HindIII and BamHI sites of C21-HCMV Kappa expression vector which was created during construction of the humanised anti-IgE antibody TESC-21 (Kolbinger et al 1993) and which originally received from M. Bendig (MRC Collaborative Centre, London, UK) (Maeda et al. 1991). The ligation products were purified by phenol/chloroform extraction, and electroporated into electrocoporation-competent Epicurian Coli®) XL1-Blue strain (Cat. N° #200228, Stratagene). After plating on LB/amp agar plates overnight at 37° C., each 12 colonies were picked to prepare plasmid DNA from a 3 ml culture using the BioRobot 9600 (Oiagen). This yielded the light chain expression vectors for the humanised antibody versions VLh and VLm, respectively, as further described in the Figures.

Human Gamma-1 Heavy Chain Expression Vectors for VHQ

For the construction of the VHQ expression vector, a step-wise approach was taken. First, the complete variable region of VHQ was assembled by PCR according to the methology as described in Kolbinger et al 1993 (Protein Eng. 1993 November; 6(8):971-80) and subcloned into the C21-HCMV-gamma-1 expression from which the C21 insert had been removed using the same enzymes. A HindIII/BamHI fragment of PCRScript clone VHQ containing the complete variable region was then subcloned into expression vector C21-HCMV-gamma-1 cleaved with the same enzymes. This yielded the final expression vector for the humanised antibody version VHQ.

Human Gamma-1 Heavy Chain Expression Vectors for VHE

The construction of the final VHE expression vector encoding for the complete humanised heavy chain of human gamma-1 isotype was achieved by directly ligating a HindIII and BamHI restricted PCR fragment encoding the variable region into the HindIII and BamHI sites of C21-HCMV gamma-1 expression vector which was created during construction of the humanised anti-IgE antibody TESC-21 (Kolbinger et al 1993) and which was also originally received from M. Bendig (MRC Collaborative Centre, London, UK) (Maeda et al. 1991).

Transient Expression in COS Cells

The following transfection protocol is adapted for adherent COS cells in 150 mm cell culture dishes, using SuperFect™ Transfection Reagent (Cat. N°301305, Qiagen). The four different expression vectors described above are used for transient transfection of cells. For expression of humanised antibody, each of two clones containing heavy chain inserts (VHE or VHQ, respectively) are co-transfected into cells with each of the two clones encoding for the light chains (humV1 or humV2, respectively), in total 4 different combinations of heavy and light chain expression vectors (VHE/humV1, VHE/humV2, VHQ/humV1 and VHQ/humV2). Before transfection, the plasmids are linearized with the restriction endonuclease Pvul which cleaves in the region encoding the resistance gene for ampicillin. The day before transfection, 4×10⁶ COS cells in 30 ml of fresh culture medium are seeded in 150 mm cell culture dishes. Seeding at this cell density generally yielded 80% confluency after 24 hours. On the day of transfection, four different combinations of linearized heavy- and light-chain DNA expression vectors (15 μg each) are diluted in a total volume of 900 μl of fresh medium without serum and antibiotics. 180 μl of SuperFect Transfection Reagent is then mixed thoroughly with the DNA solution. The DNA mixture is incubated for 10 min at room temperature to allow complex formation. While complex formation takes place, the growth medium is removed from COS cell cultures, and cells are washed once with PBS. 9 ml of fresh culture medium (containing 10% FBS and antibiotics) are then added to each reaction tube containing the transfection complexes and well mixed. The final preparation is immediately transferred to each of 4 cultures to be transfected and gently mixed. Cell cultures are then incubated with the DNA complexes for 3 hours at 37° C. and 5% CO2. After incubation, the medium containing transfection complexes is removed and replaced with 30 ml of fresh culture medium. At 48 hr post transfection, the culture supernatants are harvested.

Concentration of Culture Supernatants

For ELISA and FACS analysis, the culture supernatants collected from COS cells transfected with heavy- and light-chain plasmids are concentrated as follows. 10 ml of each supernatant are added to Centriprep YM-50 Centrifugal Filter Devices (Cat. N° 4310, Millipore) as described by the manufacturer. The Centriprep filters are centrifuged for 10 min at 3000 rpm at room temperature. The centrifugation step is then repeated again with the remaining 20 ml of supernatant using only 5 min of centrifugation and supervising the concentration evolution. The intermediate 500 μl of concentrated supernatant is recovered, transferred to new Microcon Centrifugal Filter Devices (Cat. N° 42412, Microcon) and further concentrated following the manufacturers protocol. The concentrated supernatants are centrifuged four times for 24 min at 3000 rpm at room temperature, one time for 10 min at 6000 rpm and then, three times for 5 min, always supervising the concentration evolution. The final volume of concentrated conditioned medium achieved is 100-120 μl corresponding to a 250 to 300-fold concentration of original culture medium and is stored at 4° C. until use. For comparison and control, culture medium from untransfected cells is similarly concentrated, using the same centrifugation protocol described above.

### Example 5

### Determination of Recombinant Human IgG Expression by ELISA

To determine IgG concentrations of recombinant human antibody expressed in the culture supernatants, a sandwich ELISA protocol has been developed and optimized using human IgG as standard. Flat bottom 96-well microtiter plates (Cat. N° 4-39454, Nunc Immunoplate Maxisorp) are coated overnight at 4° C. with 100 μg of goat anti-human IgG (whole molecule, Cat. N° I1011, SIGMA) at the final concentration of 0.5 μg/ml in PBS. Wells are then washed 3 times with washing buffer (PBS containing 0.05% Tween 20) and blocked for 1.5 hours at 37° C. with blocking buffer (0.5% BSA in PBS). After 3 washing cycles, the antibody samples and the standard human IgG (Cat.No. 14506, SIGMA) are prepared by serial 1.5-fold dilution in blocking buffer. 100 μl of diluted samples or standard are transfered in duplicate to the coated plate and incubated for 1 hour at room temperature. After incubation, the plates are washed 3 times with washing buffer and subsequently incubated for 1 hour with 100 μl of horseradish peroxidase-conjugated goat anti-human IgG kappa-light chain (Cat. N° A-7164, SIGMA) diluted at 1/4000 in blocking buffer. Control wells received 100 μl of blocking buffer or concentrated normal culture medium. After washing, the calorimetric quantification of bound peroxidase in the sample and standard wells is performed, using a TMB Peroxidase EIA Substrate Kit (Cat. N° 172-1067, Bio-Rad) according to the manufacturer's instructions.

The peroxidase mixture is added at 100 μl per well and incubated for 30 min at room temperature in the dark. The calorimetric reaction is stopped by addition of 100 μl of 1 M sulfuric acid and the absorbance in each well is read at 450 nm, using an ELISA plate reader (Model 3350-UV, BioRad).

With a correlation coefficient of 0.998 for the IgG standard curve, the following concentrations are determined for the four different culture concentrates (ca. 250-300 fold concentrated):

VHE/humV1 supernatant=8.26 μg/ml

VHE/humV2 supernatant=6.27 μg/ml

VHQ/humV1 supernatant=5.3 μg/ml

VHQ/humV2 supernatant=5.56 μg/ml

### Example 6

### FACS Competition Analysis (Binding Affinity)

The human T-cell line PEER is chosen as the target cell for FACS analysis because it expressed the CD45 antigen on its cell surface. To analyze the binding affinity of humanised antibody supernatants, competition experiments using FITC-labeled chimeric antibody as a reference are performed and compared with the inhibition of purified mouse antibody and of chimeric antibody. PEER cell cultures are centrifuged for 10 seconds at 3000 rpm and the medium is removed. Cells are resuspended in FACS buffer (PBS containing 1% FBS and 0.1% sodium azide) and seeded, into 96-well round-bottom microtitter plate at a cell density of 1×10⁵ cells per well. The plate is centrifuged and the supernatant is discarded. For blocking studies, 25 μl of concentrated untransfected medium or isotype matched control antibody (negative controls), unlabeled mouse antibody or chimeric antibody (positive controls) as well as concentrated supernatant containing the various combinations of humanised antibody (samples), is first added in each well at the indicated concentrations in the text. After 1 hour of incubation at 4° C., PEER cells are washed with 200 μl of FACS buffer by centrifugation. Cells are subsequently incubated for 1 hour at 4° C. with chimeric antibody conjugated with FITC in 25 μl of FACS buffer at the final concentration of 20 μg/ml. Cells are washed and resuspended in 300 μl of FACS buffer containing 2 μg/ml propidium iodide which allows gating of viable cells. The cell preparations are analyzed on a flow cytometer (FACSCalibur, Becton Dickinson). FACS analysis indicates dose-dependent blockade of fluorochrome-lab led chimeric antibody by the concentrated humanised antibody culture supernatants. No dose-dependent blockade of chimeric antibody binding is seen with the isotype matched control antibody, indicating that the blocking effect by the different humanised antibody combinations is epitope specific and that epitope specificity appears to be retained after the humanisation process.

### Example 7

### Biological Activities of CD45RB/RO Binding Molecules

In this study, we have addressed whether CD45RB/RO binding chimeric antibody, when present in cultures of polyclonally activated primary human. T cells (i) supports the differentiation of T cells with a characteristic Treg phenotype, (ii) prevents or enhances apoptosis following T cell activation, and (iii) affects expression of subset-specific antigens and receptors after restimulation.

CD45RB/RO Binding Chimeric Antibody Enhances Cell Death in Polyclonally Activated T Cells

Primary T cells (mixture of CD4+ and CD8+ T subsets) were subjected to activation by anti-CD3 plus anti-CD28 mAb (200 ng/ml each) in the presence or absence (=control) of CD45RB/RO binding chimeric antibody. Excess antibodies were removed by washing on day 2.7-amino-actinomycin D (7-AAD) as a DNA-staining dye taken up by apoptotic and necrotic cells was used to measure cell death following activation. The results show that activation of T cells in the presence of CD45RB/RO binding chimeric antibody increased the fraction of 7-AAD positive cells than two-fold on day 2 after activation. On day 7, the portion of 7-AAD positive cells was again similar in CD45RB/RO binding chimeric antibody-treated and control cultures.

CD45RB/RO Binding Chimeric Antibody but not Control mAb Treated T Cells Display a T Regulatory Cell (Treg) Phenotype

Increased expression of CD25 and the negative regulatory protein CTLA-4 (CD152) is a marker of Treg cells. Functional suppression of primary and secondary T cell responses by CD45RB/RO binding chimeric antibody may be due to the induction of Treg cells. T address this issue, T cells were activated by anti-CD3+CD28 mAbs and cultured in the presence of CD45RB/RO binding chimeric antibody or anti-LPS control mAb. The time address this issue, T cells were activated by anti-CD3+CD28 mAbs and cultured in the presence of CD45RB/RO binding chimeric antibody or anti-LPS control mAb. The time course of CTLA4 and CD25 expression reveals marked differences between controls and CD45RB/RO binding chimeric antibody-treated T cells on days 1 and 3 after secondary stimulation, indicating a Treg phenotype.

Intracellular CTLA-4 Expression is Sustained in the Presence of CD45RB/RO Binding Chimeric Antibody

It has been reported that substantial amounts of CTLA-4 can also be found intracellularly. Therefore, in parallel to surface CTLA-4 staining, intracellular CTLA-4 expression was analyzed. Moderate differences between T cell cultures were seen on day 4 after stimulation. After prolonged culture, however, high levels of intracellular CTLA-4 were sustained only in CD45RB/RO binding chimeric antibody-treated but not in control T cells.

CD45RB/RO Binding Chimeric Antibody-treated T Cells Become Double Positive for CD4 and CD8

Following stimulation, T cells induce and upregulate the expression of several surface receptors, such as CD25, CD152 (CTLA-4), CD154 (CD40-Ligand) and others. In contrast, the level of expression of C4 or CD8 is thought to stay relatively constant. We reproducibly observed a strong increase of both CD4 and CD8 antigens on CD45RB/RO binding chimeric antibody-treated but not on control Ab-treated T cells after activation. The emergence of a CD4/CD8 double-positive T cell population seems to be due to the upregulation of CD4 on the CD8+ subset and conversely CD8 on the CD4+ subset. This contrasts with a moderately low percentage of double positive T cells in control cultures.

High IL-2 Receptor Alpha-chain, but Very Low Beta-chain Expression by CD45RB/RO Binding Chimeric Antibody-treated T Cells

Treg cells are known to be constitutively positive for CD25, the IL-2 receptor alpha-chain. The regulation of other subunits of the trimeric IL-2 receptor on Treg cells is not known. Recently we have compared the expression of the beta-chain of IL-2 receptor, e.g. CD122, on T cells activated and propagated in the presence′ or′ absence of CD45RB/RO binding chimeric antibody. The results show that CD45RB/RO binding chimeric antibody-treated T cells have about ten-fold lower CD122 expression as compared to T cells in control cultures. This difference may indicate that Treg cells require factors other than IL-2 to proliferate.

### Example 8

### Sequences of the Invention (CDR Sequences of the Invention are Underlined)

### SEQ ID NO:1

Part of the Amino Acid Sequence of Chimeric Light Chain

DILLTQSPAILSVSPGERVSFSCRASQNIGTSIQWYQQRTNGSPRLLIR
SSSESISGIPSRFSGSSGSGTDFTLSINSVESEDIADYYCQQSNTWPFT
FGSGTKLEIK

### SEQ ID NO:2

Part of the Amino Acid Sequence of Chimeric Heavy Chain

EVQLQQSGPELVKPGASVKMSCKASGYTFTNYIIHWVKQEPGQGLEWIG
YFNPYNHGTKYNEKFKGRATLTADKSSNTAYMDLSSLTSEDSAIYYCAR
SGPYAWFDTWGQGTTVTVSS

### SEQ ID NO:3

Amino Acid Sequence of Chimeric Light Chain

DILLTQSPAILSVSPGERVSFSCRASQNIGTSIQWYQQRTNGSPRLLIRS
SSESISGIPSRFSGSGSGTDFTLSINSVESEDIADYYCQQSNTWPFTFGS
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### SEQ ID NO:4

Amino Acid Sequence of Chimeric Heavy Chain

EVQLQQSGPELVKPGASVKMSCKASGYTFTNYIIHWVKQEPGQGLEWIGY
FNPYNHGTKYNEKFKGRATLTADKSSNTAYMDLSSLTSEDSAIYYCARSG
PYAWFDTWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK

### SEQ ID NO:5

Nucleotide Sequence Encoding a Polypeptide of SEQ ID NO:1

GACATTCTGCTGACCCAGTCTCCAGCCATCCTGTCTGTGAGTCCAGGAGA
AAGAGTCAGTTTCTCCTGCAGGGCCAGTCAGAACATTGGCACAAGCATAC
AGTGGTATCAACAAAGAACAAATGGTTCTCCAAGGCTTCTCATAAGGTCT
TCTTCTGAGTCTATCTCTGGGATCCCTTCCAGGTTTAGTGGCAGTGGATC
AGGGACAGATTTTACTCTTAGCATCAACAGTGTGGAGTCTGAAGATATTG
CAGATTATTACTGTCAACAAAGTAATACCTGGCCATTCACGTTCGGCTCG
GGGACCAAGCTTGAAATCAAA

### SEQ ID NO:6

Nucleotide Sequence Encoding a Polypeptide of SEQ ID NO:2

GAGGTGCAGCTGCAGCAGTCAGGACCTGAACTGGTAAAGCCTGGGGCTT
CAGTGAAGATGTCCTGCAAGGCCTCTGGATACACATTCACTAATTATAT
TATCCACTGGGTGAAGCAGGAGCCTGGTCAGGGCCTTGAATGGATTGGA
TATTTTAATCCTTACAATCATGGTACTAAGTACAATGAGAAGTTCAAAG
GCAGGGCCACACTAACTGCAGACAAATCCTCCAACACAGCCTACATGGA
CCTCAGCAGCCTGACCTCTGAGGACTCTGCGATCTACTACTGTGCAAGA
TCAGGACCCTATGCCTGGTTTGACACCTGGGGCCAAGGGACCACGGTCA
CCGTCTCCTCA

### SEQ ID NO:7

Part of Amino Acid Sequence of Humanised Light Chain Designated humV2 (humV2=VLm)

DILLTQSPAT LSLSPGERAT FSCDRASQNIG TSIQWYQQKT NGAP
RLLIRS SSESISGIPSRFSGSGSGTD FTLTISSLEP EDFAVYYCSQ
Q SNTWPFTFGQ GTKLEIK

### SEQ ID NO:8

Part of Amino Acid Sequence of Humanised Light Chain Designated humV1 (humV1=VLh)

DILLTQSPAT LSLSPGERAT LSCRASQNIG TSIQWYQQKP GQ
APRLLIRS SSESISGIPSRFSGSGSGTD FTLTISSLEP EDFAVYY
CQQ SNTWPFTFGQ GTKLEIK

### SEQ ID NO:9

Part of Amino Acid Sequence of Humanised Heavy Chain Designated VHE

EVQLVESGAE VKKPGASVKV SCKASGYTFT NYIIHWVKQE PGQGLE
WIGYFNPYNHGTKY NEKFKGRATL TANKSISTAY MELSSLRSED TA
VYYCARSGPYAWFDTWGQ GTTVTVSS

### SEQ ID NO:10

Part of Amino Acid Sequence of Humanised Heavy Chain Designated VHQ

QVQLVESGAE YKKPGASVKV SCKASGYTFT NYIIHWVKQE PGQGLE
WIGYFNPYNHGTKY NEKFKGRATL TANKSISTAY MELSSLRSED TA
VYYCARSGPYAWFDTWGQ GTTVTVSS

### SEQ ID NO:11

Nucleotide Sequence Encoding Amino Acid Sequence SEQ ID NO:9

GAGGTGCAGCTGGTGGAGTCAGGAGCCGAAGTGAAAAAGCCTGGGGCTTC
AGTGAAGGTGTCCTGCAAGGCCTCTGGATACACATTCACTAATTATATTA
TCCACTGGGTGAAGCAGGAGCCTGGTCAGGGCCTTGAATGGATTGGATAT
TTTAATCCTTACAATCATGGTACTAAGTACAATGAGAAGTTCAAAGGCAG
GGCCACACTAACTGCAAACAAATCCATCAGCACAGCCTACATGGAGCTCA
GCAGCCTGCGCTCTGAGGACACTGCGGTCTACTACTGTGCAAGATCAGGA
CCCTATGCCTGGTTTGACACCTGGGGCCAAGGGACCACGGTCACCGTCTC
CTCA

### SEQ ID NO:12

Nucleotide Sequence Encoding Amino Acid Sequence SEQ ID NO:10

CAGGTGCAGCTGGTGGAGTCAGGAGCCGAAGTGAAAAAGCCTGGGGCTTC
AGTGAAGGTGTCCTGCAAGGCCTCTGGATACACATTCACTAATTATATTA
TCCACTGGGTGAAGCAGGAGCCTGGTCAGGGCCTTGAATGGATTGGATAT
TTTAATCCTTACAATCATGGTACTAAGTACAATGAGAAGTTCAAAGGCAG
GGCCACACTAACTGCAAACAAATCCATCAGCACAGCCTACATGGAGCTCA
GCAGCCTGCGCTCTGAGGACACTGCGGTCTACTACTGTGCAAGATCAGGA
CCCTATGCCTGGTTTGACACCTGGGGCCAAGGGACCACGGTCACCGTCTC
CTCA

### SEQ ID NO:13

Nucleotide Sequence Encoding Amino Acid Sequence SEQ ID NO:7

GACATTCTGCTGACCCAGTCTCCAGCCACCCTGTCTCTGAGTCCAGGAGA
AAGAGCCACTTTCTCCTGCAGGGCCAGTCAGAACATTGGCACAAGCATAC
AGTGGTATCAACAAAAAACAAATGGTGCTCCAAGGCTTCTCATAAGGTCT
TCTTCTGAGTCTATCTCTGGGATCCCTTCCAGGTTTAGTGGCAGTGGATC
AGGGACAGATTTTACTCTTACCATCAGCAGTCTGGAGCCTGAAGATTTTG
CAGTGTATTACTGTCAACAAAGTAATACCTGGCCATTCACGTTCGGCCAG
GGGACCAAGCTGGAGATCAAA

### SEQ ID NO:14

Nucleotide Sequence Encoding Amino Acid Sequence SEQ ID NO:8

GACATTCTGCTGACCCAGTCTCCAGCCACCCTGTCTCTGAGTCCAGGAGA
AAGAGCCACTCTCTCCTGCAGGGCCAGTCAGAACATTGGCACAAGCATAC
AGTGGTATCAACAAAAACCAGGTCAGGCTCCAAGGCTTCTCATAAGGTCT
TCTTCTGAGTCTATCTCTGGGATCCCTTCCAGGTTTAGTGGCAGTGGATC
AGGGACAGATTTTACTCTTACCATCAGCAGTCTGGAGCCTGAAGATTTTG
CAGTGTATTACTGTCAACAAAGTAATACCTGGCCATTCACGTTCGGCCAG
GGGACCAAGCTGGAGATCAAA

### SEQ ID NO:15

Nucleotide Sequence of the Expression Vector HCMV-G1 HuAb-VHQ (Complete DNA Sequence of a Humanised Heavy Chain Expression Vector Comprising SEQ ID NO:12 (VHQ) from 3921-4274)

| | |
|---|---|
| 1 | AGCTTTTTGC AAAAGCCTAG GCCTCCAAAA AAGCCTCCTC ACTACTTCTG |
| 51 | GAATAGCTCA GAGGCCGAGG CGGCCTCGGC CTCTGCATAA ATAAAAAAAA |
| 101 | TTAGTCAGCC ATGGGGCGGA GAATGGGCGG AACTGGGCGG AGTTAGGGGC |
| 151 | GGGATGGGCG GAGTTAGGGG CGGGACTATG GTTGCTGACT AATTGAGATG |
| 201 | CATGCTTTGC ATACTTCTGC CTGCTGGGGA GCCTGGTTGC TGACTAATTG |
| 251 | AGATGCATGC TTTGCATACT TCTGCCTGCT GGGGAGCCTG GGGACTTTCC |
| 301 | ACACCCTAAC TGACACACAT TCCACAGCTG CCTCGCGCGT TTCGGTGATG |
| 351 | ACGGTGAAAA CCTCTGACAC ATGCAGCTCC CGGAGACGGT CACAGCTTGT |
| 401 | CTGTAAGCGG ATGCCGGGAG CAGACAAGCC CGTCAGGGCG CGTCAGCGGG |
| 451 | TGTTGGCGGG TGTCGGGGCG CAGCCATGAC CCAGTCACGT AGCGATAGCG |
| 501 | GAGTGTATAC TGGCTTAACT ATGCGGCATC AGAGCAGATT GTACTGAGAG |
| 551 | TGCACCATAT GCGGTGTGAA ATACCGCACA GATGCGTAAG GAGAAAATAC |
| 601 | CGCATCAGGC GCTCTTCCGC TTCCTCGCTC ACTGACTCGC TGCGCTCGGT |
| 651 | CGTTCGGCTG CGGCOAGCGG TATCAGCTCA CTCAAAGGCG GTAATACGGT |
| 701 | TATCCACAGA ATCAGGGGAT AACGCAGGAA AGAACATGTG AGCAAAAGGC |
| 751 | CAGCAAAAGG CCAGGAACCG TAAAAAGGCC GCGTtGCTGG CGTTTTTCCA |
| 801 | TAGGCTCCGC CCCCCTGACG AGCATCACAA AAATCGACGC TCAAGTCAGA |
| 851 | GGTGGCGAAA CCCGACAGGA CTATAAAGAT ACCAGGCGTT TCCCCCTGGA |
| 901 | AGCTCCCTCG TGCGCTCTCC TGTTCCGACC CTGCCGCTTA CCGGATACCT |
| 951 | GTCCGCCTTT CTCCCTTCGG GAAGCGTGGC GCTTTCTCAT AGCTCACGCT |
| 1001 | GTAGGTATCT CAGTTCGGTG TAGGTCGTTC GCTCCAAGCT GGGCTGTGTG |
| 1051 | CACGAACCCC CCGTTCAGCC CGACCGCTGC GCCTTATCCG GTAACTATCG |
| 1101 | TCTTGAGTCC AACCCGGTAA GACACGACTT ATCGCCACTG GCAGCAGCCA |
| 1151 | CTGGTAACAG GATTAGCAGA GCGAGGTATG TAGGCGGTGC TACAGAGTTC |
| 1201 | TTGAAGTGGT GGCCTAACTA CGGCTACACT AGAAGGACAG TATTTGGTAT |
| 1251 | CTGCGCTCTG CTGAAGCCAG TTACCTTCGG AAAAAGAGTT GGTAGCTCTT |
| 1301 | GATCCGGCAA ACAAACCACC GCTGGTAGCG GTGGTTTTTT TGTTTGCAAG |
| 1351 | CAGCAGATTA CGCGCAGAAA AAAAGGATCT CAAGAAGATC CTTTGATCTT |
| 1401 | TTCTACGGGG TCTGACGCTC AGTGGAACGA AAACTCACGT TAAGGGATTT |
| 1451 | TGGTCATGAG ATTATCAAAA AGGATCTTCA CCTAGATCCT TTTAAATTAA |
| 1501 | AAATGAAGTT TTAAATCAAT CTAAAGTATA TATGAGTAAA CTTGGTCTGA |
| 1551 | CAGTTACCAA TGCTTAATCA GTGAGGCACC TATCTCAGCG ATCTGTCTAT |
| 1601 | TTCGTTCATC CATAGTTGCC TGACTCCCCG TCGTGTAGAT AACTACGATA |
| 1651 | CGGGAGGGCT TACCATCTGG CCCCAGTGCT GCAATGATAC CGCGAGACCC |
| 1701 | ACGCTCACCG GCTCCAGATT TATCAGCAAT AAACCAGCCA GCCGGAAGGG |
| 1751 | CCGAGCGCAG AAGTGGTCCT GCAACTTTAT CCGCCTCCAT CCAGTCTATT |
| 1801 | AATTGTTGCC GGGAAGCTAG AGTAAGTAGT TCGCCAGTTA ATAGTTTGCG |
| 1851 | CAACGTTGTT GCCATTGCTG CAGGCATCGT GGTGTCACGC TCGTCGTTTG |
| 1901 | GTATGGCTTC ATTCAGCTCC GGTTCCCAAC GATCAAGGCG AGTTACATGA |
| 1951 | TCCCCCATGT TGTGCAAAAA AGCGGTTAGC TCCTTCGGTC CTCCGATCGT |
| 2001 | TGTCAGAAGT AAGTTGGCCG CAGTGTTATC ACTCATGGTT ATGGCAGCAC |
| 2051 | TGCATAATTC TCTTACTGTC ATGCCATCCG TAAGATGCTT TTCTGTGACT |
| 2101 | GGTGAGTACT CAACCAAGTC ATTCTGAGAA TAGTGTATGC GGCGACCGAG |
| 2151 | TTGCTCTTGC CCGGCGTCAA CACGGGATAA TACCGCGCCA CATAGCAGAA |
| 2201 | CTTTAAAAGT GCTCATCATT GGAAAACGTT CTTCGGGGCG AAAACTCTCA |
| 2251 | AGGATCTTAC CGCTGTTGAG ATCCAGTTCG ATGTAACCCA CTCGTGCACC |
| 2301 | CAACTGATCT TCAGCATCTT TTACTTTCAC CAGCGTTTCT GGGTGAGCAA |
| 2351 | AAACAGGAAG GCAAAATGCC GCAAAAAAGG GAATAAGGGC GACACGGAAA |
| 2401 | TGTTGAATAC TCATACTCTT CCTTTTTCAA TATTATTGAA GCATTTATCA |
| 2451 | GGGTTATTGT CTCATGAGCG GATACATATT TGAATGTATT TAGAAAAATA |
| 2501 | AACAAATAGG GGTTCCGCGC ACATTTCCCC GAAAAGTGCC ACCTGACGTC |
| 2551 | TAAGAAACCA TTATTATCAT GACATTAACC TATAAAAATA GGCGTATCAC |
| 2601 | GAGGCCCTTT CGTCTTCAAG AATTCAGCTT GGCTGCAGTG AATAATAAAA |
| 2651 | TGTGTGTITG TCCGAAATAC GCGTTTTGAG ATTTCTGTCG CCGACTAAAT |
| 2701 | TCATGTCGCG CGATAGTGGT GTTTATCGCC GATAGAGATG GCGATATTGG |
| 2751 | AAAAATCGAT ATTTGAAAAT ATGGCATATT GAAAATGTCG CCGATGTGAG |
| 2801 | TTTCTGTGTA ACTGATATCG CCATTTTTCC AAAAGTGATT TTTGGGCATA |
| 2851 | CGCGATATCT GGCGATAGCG CTTATATCGT TTACGGGGGA TGGCGATAGA |
| 2901 | CGACTTTGGT GACTTGGGCG ATTCTGTGTG TCGCAAATAT CGCAGTTTCG |
| 2951 | ATATAGGTGA CAGACGATAT GAGGCTATAT CGCCGATAGA GGCGACATCA |
| 3001 | AGCTGGCACA TGGCCAATGC ATATCGATCT ATACATTGAA TCAATATTGG |
| 3051 | CCATTAGCCA TATTATTCAT TGGTTATATA GCATAAATCA ATATTGGCTA |
| 3101 | TTGGCCATTG CATACGTTGT ATCCATATCA TAATATGTAC ATTTATATTG |
| 3151 | GCTCATGTCC AACATTACCG CCATGTTGAC ATTGATTATT GACTAGTTAT |
| 3201 | TAATAGTAAT CAATTACGGG GTCATTAGTT CATAGCCCAT ATATGGAGTT |
| 3251 | CCGCGTTACA TAACTTACGG TAAATGGCCC GCCTGGCTGA CCGCCCAACG |
| 3301 | ACCCCCGCCC ATTGACGTCA ATAATGACGT ATGTTCCCAT AGTAACGCCA |
| 3351 | ATAGGGACTT TCCATTGACG TCAATGGGTG GAGTATTTAC GGTAAACTGC |
| 3401 | CCACTTGGCA GTACATCAAG TGTATCATAT GCCAAGTACG CCCCCTATTG |
| 3451 | ACGTCAATGA CGGTAAATGG CCCGCCTGGC ATTATGCCCA GTACATGACC |
| 3501 | TTATGGGACT TTCCTACTTG GCAGTACATC TACGTATTAG TCATCGCTAT |
| 3551 | TACCATGGTG ATQCGGTTTT GGCAGTACAT CAATGGGCGT GGATAGCGGT |
| 3601 | TTGACTCACG GGQATTTCCA AGTCTCCACC CCATTGACGT CAATGGGAGT |
| 3651 | TTGTTTTGGC ACCAAAATCA ACGGGACTTT CCAAAATGTC GTAACAACTC |
| 3701 | CGCCCCATTG ACGCAAATGG GCGGTAGGCG TGTACGGTGG GAGGTCTATA |
| 3751 | TAAGCAGAGC TCGTTTAGTG AACCGTCAGA TCGCCTGGAG ACGCCATCCA |
| 3801 | CGCTGTTTTG ACCTCCATAG AAGACACCGG GACCGATCCA GCCTCCGCAA |
| 3851 | GCTTGCCGCC ACCATGGACT GGACCTGGAG GGTGTTCTGC CTGCTGGCCG |
| 3901 | TGGCCCCCGG CGCCCACAGC CAGGTGCAGC TGGTGGAGTC AGGAGCCGAA |
| 3951 | GTGAAAAAGC CTGGGGCTTC AGTGAAGGTG TCCTGCAAGG CCTCTGGATA |
| 4001 | CACATTCACT AATTATATTA TCCACTGGGT GAAGCAGGAG CCTGGTCAGG |
| 4051 | GCCTTGAATG GATTGGATAT TTTAATCCTT ACAATCATGG TACTAAGTAC |
| 4101 | AATGAGAAGT TCAAAGGCAG GGCCACACTA ACTGCAAACA AATCCATCAG |
| 4151 | CACAGCCTAC ATGGAGCTCA GCAGCCTGCG CTCTGAGGAC ACTGCGGTCT |
| 4201 | ACTACTGTGC AAGATCAGGA CCCTATGCCT GGTTTGACAC CTGGGGCCAA |
| 4251 | GGGACCACGG TCACCGTCTC CTCAGGTGAG TTCTAGAAGG ATCCCAAGCT |
| 4301 | AGCTTTCTGG GGCAGGCCAG GCCTGACCTT GGCTTTGGGG CAGGGAGGGG |
| 4351 | GCTAAGGTGA GGCAGGTGGC GCCAGCCAGG TGCACACCCA ATGCCCATGA |
| 4401 | GCCCAGACAC TGGACGCTGA ACCTCGCGGA CAGTTAAGAA CCCAGGGGCC |
| 4451 | TCTGCGCCCT GGGCCCAGCT CTGTCCCACA CCGCGGTCAC ATGGCACCAC |
| 4501 | CTCTCTTGCA GCCTCCACCA AGGGCCCATC GGTCTTCCCC CTGGCACCCT |
| 4551 | CCTCCAAGAG CACCTCTGGG GGCACAGCGG CCCTGGGCTG CCTGGTCAAG |
| 4601 | GACTACTTCC CCGAACCGGT GACGGTGTCG TGGAACTCAG GCGCCCTGAC |
| 4651 | CAGCGGCGTG CACACCTTCC CGGCTGTCCT ACAGTCCTCA GGACTCTACT |
| 4701 | CCCTCAGCAG CGTGGTGACC GTGCCCTCCA GCAGCTTGGG CACCCAGACC |
| 4751 | TACATCTGCA ACGTGAATCA CAAGCCCAGC AACACCAAGG TGGACAAGAA |
| 4801 | AGTTGGTGAG AGGCCAGCAC AGGGAGGGAG GGTGTCTGCT GGAAGCCAGG |
| 4851 | CTCAGCGCTC CTGCCTGGAC GCATCCCGGC TATGCAGCCC CAGTCCAGGG |
| 4901 | CAGCAAGGCA GGCCCCGTCT GCCTCTTCAC CCGGAGGCCT CTGCCCGCCC |
| 4951 | CACTCATGCT CAGGGAGAGG GTCTTCTGGC TTTTTCCCCA GGCTCTGGGC |
| 5001 | AGGCACAGGC TAGGTGCCCC TAACCCAGGC CCTGCACACA AAGGGGCAGG |
| 5051 | TGCTGGGCTC AGACCTGCCA AGAGCCATAT CCGGGAGGAC CCTGCCCCTG |
| 5101 | ACCTAAGCCC ACCCCAAAGG CCAAACTCTC CACTCCCTCA GCTCGGACAC |
| 5151 | CTTCTCTCCT CCCAGATTCC AGTAACTCCC AATCTTCTCT CTGCAGAGCC |
| 5201 | CAAATCTTGT GACAAAACTC ACACATGCCC ACCGTGCCCA GGTAAGCCAG |
| 5251 | CCCAGGCCTC GCCCTCCAGC TCAAGGCGGG ACAGGTGCCC TAGAGTAGCC |
| 5301 | TGCATCCAGG GACAGGCCCC AGCCGGGTGC TGACACGTCC ACCTCCATCT |
| 5351 | CTTCCTCAGC ACCTGAACTC CTGGGGGGAC CGTCAGTCTT CCTCTTCCCC |
| 5401 | CCAAAACCCA AGGACACCCT CATGATCTCC CGGACCCCTG AGGTCACATG |
| 5451 | CGTGGTGGTG GACGTGAGCC ACGAAGACCC TGAGGTCAAG TTCAACTGGT |
| 5501 | ACGTGGACGG CGTGGAGGTG CATAATGCCA AGACAAAGCC GCGGGAGGAG |
| 5551 | CAGTACAACA GCACGTACCG TGTGGTCAGC GTCCTCACCG TCCTGCACCA |
| 5601 | GGACTGGCTG AATGGCAAGG AGTACAAGTG CAAGGTCTCC AACAAAGCCC |
| 5651 | TCCCAGCCCC CATCGAGAAA ACCATCTCCA AAGCCAAAGG TGGGACCCGT |
| 5701 | GGGGTGCGAG GGCCACATGG ACAGAGGCCG GCTCGGCCCA CCCTCTGCCC |
| 5751 | TGAGAGTGAC CGCTGTACCA ACCTCTGTCC CTACAGGGCA GCCCCGAGAA |
| 5801 | CCACAGGTGT ACACCCTGCC CCCATCCCGG GATGAGCTGA CCAAGAACCA |
| 5851 | GGTCAGCCTG ACCTGCCTGG TCAAAGGCTT CTATCCCAGC GACATCGCCG |
| 5901 | TGGAGTGGGA GAGCAATGGG CAGCCGGAGA ACAACTACAA GACCACGCCT |
| 5951 | CCCGTGCTGG ACTCCGACGG CTCCTTCTTC CTCTACAGCA AGCTCACCGT |
| 6001 | GGACAAGAGC AGGTGGCAGC AGGGGAACGT CTTCTCATGC TCCGTGATGC |
| 6051 | ATGAGGCTCT GCACAACCAC TACACGCAGA AGAGCCTCTC CCTGTCTCCG |
| 6101 | GGTAAATGAG TGCGACGGCC GGCAAGCCCC CGCTCCCCGG GCTCTCGCGG |
| 6151 | TCGCACGAGG ATGCTTGGCA CGTACCCCCT GTACATACTT CCCGGGCGCC |
| 6201 | CAGCATGGAA ATAAAGCACC CAGCGCTGCC CTGGGCCCCT GCGAGACTGT |
| 6251 | GATGGTTCTT TCCACGGGTC AGGCCGAGTC TGAGGCCTGA GTGGCATGAG |
| 6301 | ATCTGATATC ATCGATGAAT TCGAGCTCGG TACCCGGGGA TCGATCCAGA |
| 6351 | CATGATAAGA TACATTGATG AGTTTGGACA AACCACAACT AGAATGCAGT |
| 6401 | GAAAAAAATG CTTTATTTGT GAAATTTGTG ATGCTATTGC TTTATTTGTA |
| 6451 | ACCATTATAA GCTGCAATAA ACAAGTTAAC AACAACAATT GCATTCATTT |
| 6501 | TATGTTTCAG GTTCAGGGGG AGGTGTGGGA GGTTTTTTAA AGCAAGTAAA |
| 6551 | ACCTCTACAA ATGTGGTATG GCTGATTATG ATCTCTAGTC AAGGCACTAT |
| 6601 | ACATCAAATA TTCCTTATTA ACCCCTTTAC AAATTAAAAA GCTAAAGGTA |
| 6651 | CACAATTTTT GAGCATAGTT ATTAATAGCA GACACTCTAT GCCTGTGTGG |
| 6701 | AGTAAGAAAA AACAGTATGT TATGATTATA ACTGTTATGC CTACTTATAA |
| 6751 | AGGTTACAGA ATATTTTTCC ATAATTTTCT TGTATAGCAG TGCAGCTTTT |
| 6801 | TCCTTTGTGG TGTAAATAGC AAAGCAAGCA AGAGTTCTAT TACTAAACAC |
| 6851 | AGCATGACTC AAAAAACTTA GCAATTCTGA AGGAAAGTCC TTGGGGTCTT |
| 6901 | CTACCTTTCT CTTCTTTTTT GGAGGAGTAG AATGTTGAGA GTCAGCAGTA |
| 6951 | GCCTCATCAT CACTAGATGG CATTTCTTCT GAGCAAAACA GGTTTTCCTC |
| 7001 | ATTAAAGGCA TTCCACCACT GCTCCCATTC ATCAGTTCCA TAGGTTGGAA |
| 7051 | TCTAAAATAC ACAAACAATT AGAATCAGTA GTTTAACACA TTATACACTT |
| 7101 | AAAAATTTTA TATTTACCTT AGAGCTTTAA ATCTCTGTAG GTAGTTTGTC |
| 7151 | CAATTATGTC ACACCACAGA AGTAAGGTTC CTTCACAAAG ATCCGGGACC |
| 7201 | AAAGCGGCCA TCGTGCCTCC CCACTCCTGC AGTTCGGGGG CATGGATGCG |
| 7251 | CGGATAGCCG CTGCTGGTTT CCTGGATGCC GACGGATTTG CACTGCCGGT |
| 7301 | AGAACTCCGC GAGGTCGTCC AGCCTCAGGC AGCAGCTGAA CCAACTCGCG |
| 7351 | AGGGGATCGA GCCCGGGGTG GGCGAAGAAC TCCAGCATGA GATCCCCGCG |
| 7401 | CTGGAGGATC ATCCAGCCGG CGTCCCGGAA AACGATTCCG AAGCCCAACC |
| 7451 | TTTCATAGAA GGCGGCGGTG GAATCGAAAT CTCGTGATGG CAGGTTGGGC |
| 7501 | GTCGCTTGGT CGGTCATTTC GAACCCCAGA GTCCCGCTCA GAAGAACTCG |
| 7551 | TCAAGAAGGC GATAGAAGGC GATGCGCTGC GAATCGGGAG CGGCGATACC |
| 7601 | GTAAAGCACQ AGGAAGCGGT CAGCCCATTC GCCGCCAAGC TCTTCAGCAA |
| 7651 | TATCACGGGT AQCCAACGCT ATGTCCTGAT AGCGGTCCGC CACACCCAGC |
| 7701 | CGGCCACAGT CGATGAATCC AGAAAAGCGG CCATTTTCCA CCATGATATT |
| 7751 | CGGCAAGCAG GCATCGCCAT GGGTCACGAC GAGATCCTCG CCGTCGGGCA |
| 7801 | TGCGCGCCTT GAGCCTGGCG AACAGTTCGG CTGGCGCGAG CCCCTGATGC |
| 7851 | TCTTCGTCCA GATCATCCTG ATCGACAAGA CCGGCTTCCA TCCGAGTACG |
| 7901 | TGCTCGCTCG ATGCGATGTT TCGCTTGGTG GTCGAATGGG CAGGTAGCCG |
| 7951 | GATCAAGCGT ATGCAGCCGC CGCATTGCAT CAGCCATGAT GGATACTTTC |
| 8001 | TCGGCAGGAG CAAGGTGAGA TGACAGGAGA TCCTGCCCCG GCACTTCGCC |
| 8051 | CAATAGCAGC CAGTCCCTTC CCGCTTCAGT GACAACGTCG AGCACAGCTG |
| 8101 | CGCAAGGAAC GCCCGTCGTG GCCAGCCACG ATAGCCGCGC TGCCTCGTCC |
| 8151 | TGCAGTTCAT TCAGGGCACC GGACAGGTCG GTCTTGACAA AAAGAACCGG |
| 8201 | GCGCCCCTGC GCTGACAGCC GGAACACGGC GGCATCAGAG CAGCCGATTG |
| 8251 | TCTGTTGTGC CCAGTCATAG CCGAATAGCC TCTCCACCCA AGCGGCCGGA |
| 8301 | GAACCTGCGT GCAATCCATC TTGTTCAATC ATGCGAAACG ATCCTCATCC |
| 8351 | TGTCTCTTGA TCAGATCTTG ATCCCCTGCG CCATCAGATC CTTGGCGGCA |
| 8401 | AGAAAGCCAT CCAGTTTACT TTGCAGGGCT TCCCAACCTT ACCAGAGGGC |
| 8451 | GCCCCAGCTG GCAATTCCGG TTCGCTTGCT GTCCATAAAA CCGCCCAGTC |
| 8501 | TAGCTATCGC CATGTAAGCC CACTGCAAGC TACCTGCTTT CTCTTTGCGC |
| 8551 | TTGCGTTTTC CCTTGTCCAG ATAGCCCAGT AGCTGACATT CATCCGGGGT |
| 8601 | CAGCACCGTT TCTGCGGACT GGCTTTCTAC GTGTTCCGCT TCCTTTAGCA |
| 8651 | GCCCTTGCGC CCTGAGTGCT TGCGGCAGCG TGAAGCT |

### SEQ ID NO:16

Nucleotide Sequence of the Expression Vector HCMV-G1 HuAb-VHE (Complete DNA Sequence of a Humanised Heavy Chain Expression Vector Comprising SEQ ID NO: 11 (VHE) from 3921-4274)

| | |
|---|---|
| 1 | AGCTTTTTGC AAAAGCCTAG GCCTCCAAAA AAGCCTCCTC ACTACTTCTG |
| 51 | GAATAGCTCA GAGGCCGAGG CGGCCTCGGC CTCTGCATAA ATAAAAAAAA |
| 101 | TTAGTCAGCC ATGGGGCGGA GAATGGGCGG AACTGGGCGG AGTTAGGGGC |
| 151 | GGGATGGGCG GAGTTAGGGG CGGGACTATG GTTGCTGACT AATTGAGATG |
| 201 | CATGCTTTGC ATACTTCTGC CTGCTGGGGA GCCTGGTTGC TGACTAATTG |
| 251 | AGATGCATGC TTTGCATACT TCTGCCTGCT GGGGAGCCTG GGGACTTTCC |
| 301 | ACACCCTAAC TGACACACAT TCCACAGCTG CCTCGCGCGT TTCGGTGATG |
| 351 | ACGGTGAAAA CCTCTGACAC ATGCAGCTCC CGGAGACGGT CACAGCTTGT |
| 401 | CTGTAAGCGG ATGCCGGGAG CAGACAAGCC CGTCAGGGCG CGTCAGCGGG |
| 451 | TGTTGGCGGG TGTCGGGGCG CAGCCATGAC CCAGTCACGT AGCGATAGCG |
| 501 | GAGTGTATAC TGGCTTAACT ATGCGGCATC AGAGCAGATT GTACTGAGAG |
| 551 | TGCACCATAT GCGGTGTGAA ATACCGCACA GATGCGTAAG GAGAAAATAC |
| 601 | CGCATCAGGC GCTCTTCCGC TTCCTCGCTC ACTGACTCGC TGCGCTCGGT |
| 651 | CGTTCGGCTG CGGCGAGCGG TATCAGCTCA CTCAAAGGCG GTAATACGGT |
| 701 | TATCCACAGA ATCAGGGGAT AACGCAGGAA AGAACATGTG AGCAAAAGGC |
| 751 | CAGCAAAAGG CCAGGAACCG TAAAAAGGCC GCGTTGCTGG CGTTTTTCCA |
| 801 | TAGGCTCCGC CCCCCTGACG AGCATCACAA AAATCGACGC TCAAGTCAGA |
| 851 | GGTGGCGAAA CCCGACAGGA CTATAAAGAT ACCAGGCGTT TCCCCCTGGA |
| 901 | AGCTCCCTCG TGCGCTCTCC TGTTCCGACC CTGCCGCTTA CCGGATACCT |
| 951 | GTCCGCCTTT CTCCCTTCGG GAAGCGTGGC GCTTTCTCAT AGCTCACGCT |
| 1001 | GTAGGTATCT CAGTTCGGTG TAGGTCGTTC GCTCCAAGCT GGGCTGTGTG |
| 1051 | CACGAACCCC CCGTTCAGCC CGACCGCTGC GCCTTATCCG GTAACTATCG |
| 1101 | TCTTGAGTCC AACCCGGTAA GACACGACTT ATCGCCACTG GCAGCAGCCA |
| 1151 | CTGGTAACAG GATTAGCAGA GCQAGGTATG TAGGCGGTGC TACAGAGTTC |
| 1201 | TTGAAGTGGT GGCCTAACTA CGGCTACACT AGAAGGACAG TATTTGGTAT |
| 1251 | CTGCGCTCTG CTGAAGCCAG TTACCTTCGG AAAAAGAGTT GGTAGCTCTT |
| 1301 | GATCCGGCAA ACAAACCACC GCTGGTAGCG GTGGTTTTTT TGTTTGCAAG |
| 1351 | CAGCAGATTA CGCGCAGAAA AAAAGGATCT CAAGAAGATC CTTTGATCTT |
| 1401 | TTCTACGGGG TCTGACGCTC AGTGGAACGA AAACTCACGT TAAGGGATTT |
| 1451 | TGGTCATGAG ATTATCAAAA AGGATCTTCA CCTAGATCCT TTTAAATTAA |
| 1501 | AAATGAAGTT TTAAATCAAT CTAAAGTATA TATGAGTAAA CTTGGTCTGA |
| 1551 | CAGTTACCAA TGCTTAATCA GTGAGGCACC TATCTCAGCG ATCTGTCTAT |
| 1601 | TTCGTTCATC CATAGTTGCC TGACTCCCCG TCGTGTAGAT AACTACGATA |
| 1651 | CGGGAGGGCT TACCATCTGG CCCCAGTGCT GCAATGATAC CGCGAGACCC |
| 1701 | ACGCTCACCG GCTCCAGATT TATCAGCAAT AAACCAGCCA GCCGGAAGGG |
| 1751 | CCGAGCGCAG AAGTGGTCCT GCAACTTTAT CCGCCTCCAT CCAGTCTATT |
| 1801 | AATTGTTGCC GGGAAGCTAG AGTAAGTAGT TCGCCAGTTA ATAGTTTGCG |
| 1851 | CAACGTTGTT GCCATTGCTG CAGGCATCGT GGTGTCACGC TCGTCGTTTG |
| 1901 | GTATGGCTTC ATTCAGCTCC GGTTCCCAAC GATCAAGGCG AGTTACATGA |
| 1951 | TCCCCCATGT TGTGCAAAAA AGCGGTTAGC TCCTTCGGTC CTCCGATCGT |
| 2001 | TGTCAGAAGT AAGTTGGCCG CAGTGTTATC ACTCATGGTT ATGGCAGCAC |
| 2051 | TGCATAATTC TCTThCTGTC ATGCCATCCG TAAGATGCTT TTCTGTGACT |
| 2101 | GGTGAGTACT CAACCAAGTC ATTCTGAGAA TAGTGTATGC GGCGACCGAG |
| 2151 | TTGCTCTTGC CCGGCGTCAA CACGGGATAA TACCGCGCCA CATAGCAGAA |
| 2201 | CTTTAAAAGT GCTCATCATT GGAAAACGTT CTTCGGGGCG AAAACTCTCA |
| 2251 | AGGATCTTAC CGCTGTTGAG ATCCAGTTCG ATGTAACCCA CTCGTGCACC |
| 2301 | CAACTGATCT TCAGCATCTT TTACTTTCAC CAGCGTTTCT GGGTGAGCAA |
| 2351 | AAACAGGAAG GCAAAATGCC GCAAAAAAGG GAATAAGGGC GACACGGAAA |
| 2401 | TGTTGAATAC TCATACTCTT CCTTTTTCAA TATTATTGAA GCATTTATCA |
| 2451 | GGGTTATTGT CTCATGAGCG GATACATATT TGAATGTATT TAGAAAAATA |
| 2501 | AACAAATAGG GGTTCCGCGC ACATTTCCCC GAAAAGTGCC ACCTGACGTC |
| 2551 | TAAGAAACCA TTATTATCAT GACATTAACC TATAAAAATA GGCGTATCAC |
| 2601 | GAGGCCCTTT CGTCTTCAAG AATTCAGCTT GGCTGCAGTG AATAATAAAA |
| 2651 | TGTGTGTTTG TCCGAAATAC GCGTTTTGAG ATTTCTGTCG CCGACTAAAT |
| 2701 | TCATGTCGCG CGATAGTGGT GTTTATCGCC GATAGAGATG GCGATATTGG |
| 2751 | AAAAATCGAT ATTTGAAAAT ATGGCATATT GAAAATGTCG CCGATGTGAG |
| 2801 | TTTCTGTGTA ACTGATATCG CCATTTTTCC AAAAGTGATT TTTGGGCATA |
| 2851 | CGCGATATCT GGCGATAGCG CTTATATCGT TTACGGGGGA TGGCGATAGA |
| 2901 | CGACTTTGGT GACTTGGGCG ATTCTGTGTG TCGCAAATAT CGCAGTTTCG |
| 2951 | ATATAGGTGA CAGACGATAT GAGGCTATAT CGCCGATAGA GGCGACATCA |
| 3001 | AGCTGGCACA TGGCCAATGC ATATCGATCT ATACATTGAA TCAATATTGG |
| 3051 | CCATTAGCCA TATTATTCAT TGGTTATATA GCATAAATCA ATATTGGCTA |
| 3101 | TTGGCCATTG CATACGTTGT ATCCATATCA TAATATGTAC ATTTATATTG |
| 3151 | GCTCATGTCC AACATTACCG CCATGTTGAC ATTGATTATT GACTAGTTAT |
| 3201 | TAATAGTAAT CAATTACGGG GTCATTAGTT CATAGCCCAT ATATGGAGTT |
| 3251 | CCGCGTTACA TAACTTACGG TAAATGGCCC GCCTGGCTGA CCGCCCAACG |
| 3301 | ACCCCCGCCC ATTGACGTCA ATAATGACGT ATGTTCCCAT AGTAACGCCA |
| 3351 | ATAGGGACTT TCCATTGACG TCAATGGGTG GAGTATTTAC GGTAAACTGC |
| 3401 | CCACTTGGCA GTACATCAAG TGTATCATAT GCCAAGTACG CCCCCTATTG |
| 3451 | ACGTCAATGA CGGTAAATGG CCCGCCTGGC ATTATGCCCA GTACATGACC |
| 3501 | TTATGGGACT TTCCTACTTG GCAGTACATC TACGTATTAG TCATCGCTAT |
| 3551 | TACCATGGTG ATGCGGTTTT GGCAGTACAT CAATGGGCGT GGATAGCGGT |
| 3601 | TTGACTCACG GGGATTTCCA AGTCTCCACC CCATTGACGT CAATGGGAGT |
| 3651 | TTGTTTTGGC ACCAAAATCA ACGGGACTTT CCAAAATGTC GTAACAACTC |
| 3701 | CGCCCCATTG ACGCAAATGG GCGGTAGGCG TGTACGGTGG GAGGTCTATA |
| 3751 | TAAGCAGAGC TCGTTTAGTG AACCGTCAGA TCGCCTGGAG ACGCCATCCA |
| 3801 | CGCTGTTTTG ACCTCCATAG AAGACACCGG GACCGATCCA GCCTCCGCAA |
| 3851 | GCTTGCCGCC ACCATGGACT GGACCTGGAG GGTGTTCTGC CTGCTGGCCG |
| 3901 | TGGCCCCCGG CGCCCACAGC GAGGTGCAGC TGGTGGAGTC AGGAGCCGAA |
| 3951 | GTGAAAAAGC CTGGGGCTTC AGTGAAGGTG TCCTGCAAGG CCTCTGGATA |
| 4001 | CACATTCACT AATTATATTA TCCACTGGGT GAAGCAGGAG CCTGGTCAGG |
| 4051 | GCCTTGAATG GATTGGATAT TTTAATCCTT ACAATCATGG TACTAAGTAC |
| 4101 | AATGAGAAGT TCAAAGGCAG GGCCACACTA ACTGCAAACA AATCCATCAG |
| 4151 | CACAGCCTAC ATGGAGCTCA GCAGCCTGCG CTCTGAGGAC ACTGCGGTCT |
| 4201 | ACTACTGTGC AAGATCAGGA CCCTATGCCT GGTTTGACAC CTGGGGCCAA |
| 4251 | GGGACCACGG TCACCGTCTC CTCAGGTGAG TTCTAGAAGG ATCCCAAGCT |
| 4301 | AGCTTTCTGG GGCAGGCCAG GCCTGACCTT GGCTTTGGGG CAGGGAGGGG |
| 4351 | GCTAAGGTGA GGCAGGTGGC GCCAGCCAGG TGCACACCCA ATGCCCATGA |
| 4401 | GCCCAGACAC TGGACGCTGA ACCTCGCGGA CAGTTAAGAA CCCAGGCGCC |
| 4451 | TCTGCGCCCT GGGCCCAGCT CTGTCCCACA CCGCGGTCAC ATGGCACCAC |
| 4501 | CTCTCTTGCA GCCTCCACCA AGGGCCCATC GGTCTTCCCC CTGGCACCCT |
| 4551 | CCTCCAAGAG CACCTCTGGG GGCACAGCGG CCCTGGGCTG CCTGGTCAAG |
| 4601 | GACTACTTCC CCGAACCGGT GACGGTGTCG TGGAACTCAG GCGCCCTGAC |
| 4651 | CAGCGGCGTG CACACCTTCC CGGCTGTCCT ACAGTCCTCA GGACTCTACT |
| 4701 | CCCTCAGCAG CGTGGTGACC GTGCCCTCCA GCAGCTTGGG CACCCAGACC |
| 4751 | TACATCTGCA ACGTGAATCA CAAGCCCAGC AACACCAAGG TGGACAAGAA |
| 4801 | AGTTGGTGAG AGGCCAGCAC AGGGAGGGAG GGTGTCTGCT GGAAGCCAGG |
| 4851 | CTCAGCGCTC CTGCCTGGAC GCATCCCGGC TATGCAGCCC CAGTCCAGGG |
| 4901 | CAGCAAGGCA GGCCCCGTCT GCCTCTTCAC CCGGAGGCCT CTGCCCGCCC |
| 4951 | CACTCATGCT CAGGGAGAGG GTCTTCTGGC TTTTTCCCCA GGCTCTGGGC |
| 5001 | AGGCACAGGC TAGGTGCCCC TAACCCAGGC CCTGCACACA AAGGGGCAGG |
| 5051 | TGCTGGGCTC AGACCTGCCA AGAGCCATAT CCGGGAGGAC CCTGCCCCTG |
| 5101 | ACCTAAGCCC ACCCCAAAGG CCAAACTCTC CACTCCCTCA GCTCGGACAC |
| 5151 | CTTCTCTCCT CCCAGATTCC AGTAACTCCC AATCTTCTCT CTGCAGAGCC |
| 5201 | CAAATCTTGT GACAAAACTC ACACATGCCC ACCGTGCCCA GGTAAGCCAG |
| 5251 | CCCAGGCCTC GCCCTCCAGC TCAAGGCGGG ACAGGTGCCC TAGAGTAGCC |
| 5301 | TGCATCCAGG GACAGGCCCC AGCCGGGTGC TGACACGTCC ACCTCCATCT |
| 5351 | CTTCCTCAGC ACCTGAACTC CTGGGGGGAC CGTCAGTCTT CCTCTTCCCC |
| 5401 | CCAAAACCCA AGGACACCCT CATGATCTCC CGGACCCCTG AGGTCACATG |
| 5451 | CGTGGTGGTG GACGTGAGCC ACGAAGACCC TGAGGTCAAG TTCAACTGGT |
| 5501 | ACGTGGACGG CGTGGAGGTG CATAATGCCA AGACAAAGCC GCGGGAGGAG |
| 5551 | CAGTACAACA GCACGTACCG TGTGGTCAGC GTCCTCACCG TCCTGCACCA |
| 5601 | GGACTGGCTG AATGGCAAGG AGTACAAGTG CAAGGTCTCC AACAAAGCCC |
| 5651 | TCCCAGCCCC CATCGAGAAA ACCATCTCCA AAGCCAAAGG TGGGACCCGT |
| 5701 | GGGGTGCGAG GGCCACATGG ACAGAGGCCG GCTCGGCCCA CCCTCTGCCC |
| 5751 | TGAGAGTGAC CGCTGTACCA ACCTCTGTCC CTACAGGGCA GCCCCGAGAA |
| 5801 | CCACAGGTGT ACACCCTGCC CCCATCCCGG GATGAGCTGA CCAAGAACCA |
| 5851 | GGTCAGCCTG ACCTGCCTGG TCAAAGGCTT CTATCCCAGC GACATCGCCG |
| 5901 | TGGAGTGGGA GAGCAATGGG CAGCCGGAGA ACAACTACAA GACCACGCCT |
| 5951 | CCCGTGCTGG ACTCCGACGG CTCCTTCTTC CTCTACAGCA AGCTCACCGT |
| 6001 | GGACAAGAGC AGGTGGCAGC AGGGGAACGT CTTCTCATGC TCCGTGATGC |
| 6051 | ATGAGGCTCT GCACAACCAC TACACGCAGA AGAGCCTCTC CCTGTCTCCG |
| 6101 | GGTAAATGAG TGCGACGGCC GGCAAGCCCC CGCTCCCCGG GCTCTCGCGG |
| 6151 | TCGCACGAGG ATGCTTGGCA CGTACCCCCT GTACATACTT CCCGGGCGCC |
| 6201 | CAGCATGGAA ATAAAGCACC CAGCGCTGCC CTGGGCCCCT GCGAGACTGT |
| 6251 | GATGGTTCTT TCCACGGGTC AGGCCGAGTC TGAGGCCTGA GTGGCATGAG |
| 6301 | ATCTGATATC ATCGATGAAT TCGAGCTCGG TACCCGGGGA TCGATCCAGA |
| 6351 | CATGATAAGA TACATTGATG AGTTTGGACA AACCACAACT AGAATGCAGT |
| 6401 | GAAAAAAATG CTTTATTTGT GAAATTTGTG ATGCTATTGC TTTATTTGTA |
| 6451 | ACCATTATAA GCTGCAATAA ACAAGTTAAC AACAACAATT GCATTCATTT |
| 6501 | TATGTTTCAG GTTCAGGGGG AGGTGTGGGA GGTTTTTTAA AGCAAGTAAA |
| 6551 | ACCTCTACAA ATGTGGTATG GCTGATTATG ATCTCTAGTC AAGGCACTAT |
| 6601 | ACATCAAATA TTCCTTATTA ACCCCTTTAC AAATTAAAAA GCTAAAGGTA |
| 6651 | CACAATTTTT GAGCATAGTT ATTAATAGCA GACACTCTAT GCCTGTGTGG |
| 6701 | AGTAAGAAAA AACAGTATGT TATGATTATA ACTGTTATGC CTACTTATAA |
| 6751 | AGGTTACAGA ATATTTTTCC ATAATTTTCT TGTATAGCAG TGCAGCTTTT |
| 6801 | TCCTTTGTGG TGTAAATAGC AAAGCAAGCA AGAGTTCTAT TACTAAACAC |
| 6851 | AGCATGACTC AAAAAACTTA GCAATTCTGA AGGAAAGTCC TTGGGGTCTT |
| 6901 | CTACCTTTCT CTTCTTTTTT GGAGGAGTAG AATGTTGAGA GTCAGCAGTA |
| 6951 | GCCTCATCAT CACTAGATGG CATTTCTTCT GAGCAAAACA GGTTTTCCTC |
| 7001 | ATTAAAGGCA TTCCACCACT GCTCCCATTC ATCAGTTCCA TAGGTTGGAA |
| 7051 | TCTAAAATAC ACAAACAATT AGAATCAGTA GTTTAACACA TTATACACTT |
| 7101 | AAAAATTTTA TATTTACCTT AGAGCTTTAA ATCTCTGTAG GTAGTTTGTC |
| 7151 | CAATTATGTC ACACCACAGA AGTAAGGTTC CTTCACAAAG ATCCGGGACC |
| 7201 | AAAGCGGCCA TCGTGCCTCC CCACTCCTGC AGTTCGGGGG CATGGATGCG |
| 7251 | CGGATAGCCG CTGCTGGTTT CCTGGATGCC GACGGATTTG CACTGCCGGT |
| 7301 | AGAACTCCGC GAGGTCGTCC AGCCTCAGGC AGCAGCTGAA CCAACTCGCG |
| 7351 | AGGGGATCGA GCCCGGGGTG GGCGAAGAAC TCCAGCATGA GATCCCCGCG |
| 7401 | CTGGAGGATC ATCCAGCCGG CGTCCCGGAA AACGATTCCG AAGCCCAACC |
| 7451 | TTTCATAGAA GGCGGCGGTG GAATCGAAAT CTCGTGATGG CAGGTTGGGC |
| 7501 | GTCGCTTGGT CGGTCATTTC GAACCCCAGA GTCCCGCTCA GAAGAACTCG |
| 7551 | TCAAGAAGGC GATAGAAGGC GATGCGCTGC GAATCGGGAG CGGCGATACC |
| 7601 | GTAAAGCACG AGGAAGCGGT CAGCCCATTC GCCGCCAAGC TCTTCAGCAA |
| 7651 | TATCACGGGT AGCCAACGCT ATGTCCTGAT AGCGGTCCGC CACACCCAGC |
| 7701 | CGGCCACAGT CGATGAATCC AGAAAAGCGG CCATTTTCCA CCATGATATT |
| 7751 | CGGCAAGCAG GCATCGCCAT GGGTCACGAC GAGATCCTCG CCGTCGGGCA |
| 7801 | TGCGCGCCTT GAGCCTGGCG AACAGTTCGG CTGGCGCGAG CCCCTGATGC |
| 7851 | TCTTCGTCCA GATCATCCTG ATCGACAAGA CCGGCTTCCA TCCGAGTACG |
| 7901 | TGCTCGCTCG ATGCGATGTT TCGCTTGGTG GTCGAATGGG CAGGTAGCCG |
| 7951 | GATCAAGCGT ATGCAGCCGC CGCATTGCAT CAGCCATGAT GGATACTTTC |
| 8001 | TCGGCAGGAG CAAGGTGAGA TGACAGGAGA TCCTGCCCCG GCACTTCGCC |
| 8051 | CAATAGCAGC CAGTCCCTTC CCGCTTCAGT GACAACGTCG AGCACAGCTG |
| 8101 | CGCAAGGAAC GCCCGTCGTG GCCAGCCACG ATAGCCGCGC TGCCTCGTCC |
| 8151 | TGCAGTTCAT TCAGGGCACC GGACAGGTCG GTCTTGACAA AAAGAACCGG |
| 8201 | GCGCCCCTGC GCTGACAGCC GGAACACGGC GGCATCAGAG CAGCCGATTG |
| 8251 | TCTGTTGTGC CCAGTCATAG CCGAATAGCC TCTCCACCCA AGCGGCCGGA |
| 8301 | GAACCTGCGT GCAATCCATC TTGTTCAATC ATGCGAAACG ATCCTCATCC |
| 8351 | TGTCTCTTGA TCAGATCTTG ATCCCCTGCG CCATCAGATC CTTGGCGGCA |
| 8401 | AGAAAGCCAT CCAGTTTACT TTGCAGGGCT TCCCAACCTT ACCAGAGGGC |
| 8451 | GCCCCAGCTG GCAATTCCGG TTCGCTTGCT GTCCATAAAA CCGCCCAGTC |
| 8501 | TAGCTATCGC CATGTAAGCC CACTGCAAGC TACCTGCTTT CTCTTTGCGC |
| 8551 | TTGCGTTTTC CCTTGTCCAG ATAGCCCAGT AGCTGACATT CATCCGGGGT |
| 8601 | CAGCACCGTT TCTGCGGACT GGCTTTCTAC GTGTTCCGCT TCCTTTAGCA |
| 8651 | GCCCTTGCGC CCTGAGTGCT TGCGGCAGCG TGAAGCT |

### SEQ ID NO:17

Nucleotide Sequence of the Expression Vector HCMV-K HuAb-VL1 hum V1 (Complete DNA Sequence of a Humanised Light Chain Expression Vector Comprising SEQ ID NO: 14 (humV1=VLh) from 3964-4284

| | |
|---|---|
| 1 | CTAGCTTTTT GCAAAAGCCT AGGCCTCCAA AAAAGCCTCC TCACTACTTC |
| 51 | TGGAATAGCT CAGAGGCCGA GGCGGCCTCG GCCTCTGCAT AAATAAAAAA |
| 101 | AATTAGTCAG CCATGGGGCG GAGAATGGGC GGAACTGGGC GGAGTTAGGG |
| 151 | GCGGGATGGG CGGAGTTAGG GGCGGGACTA TGGTTGCTGA CTAATTGAGA |
| 201 | TGCATGCTTT GCATACTTCT GCCTGCTGGG GAGCCTGGTT GCTGACTAAT |
| 251 | TGAGATGCAT GCTTTGCATA CTTCTGCCTG CTGGGGAGCC TGGGGACTTT |
| 301 | CCACACCCTA ACTGACACAC ATTCCACAGC TGCCTCGCGC GTTTCGGTGA |
| 351 | TGACGGTGAA AACCTCTGAC ACATGCAGCT CCCGGAGACG GTCACAGCTT |
| 401 | GTCTGTAAGC GGATGCCGGG AGCAGACAAG CCCGTCAGGG CGCGTCAGCG |
| 451 | GGTGTTGGCG GGTGTCGGGG CGCAGCCATG ACCCAGTCAC GTAGCGATAG |
| 501 | CGGAGTGTAT ACTGGCTTAA CTATGCGGCA TCAGAGCAGA TTGTACTGAG |
| 551 | AGTGCACCAT ATGCGGTGTG AAATACCGCA CAGATGCGTA AGGAGAAAAT |
| 601 | ACCGCATCAG GCGCTCTTCC GCTTCCTCGC TCACTGACTC GCTGCGCTCG |
| 651 | GTCGTTCGGC TGCGGCGAGC GGTATCAGCT CACTCAAAGG CGGTAATACG |
| 701 | GTTATCCACA GAATCAGGGG ATAACGCAGG AAAGAACATG TGAGCAAAAG |
| 751 | GCCAGCAAAA GGCCAGGAAC CGTAAAAAGG CCGCGTTGCT GGCGTTTTTC |
| 801 | CATAGGCTCC GCCCCCCTGA CGAGCATCAC AAAAATCGAC GCTCAAGTCA |
| 851 | GAGGTGGCGA AACCCGACAG GACTATAAAG ATACCAGGCG TTTCCCCCTG |
| 901 | GAAGCTCCCT CGTGCGCTCT CCTGTTCCGA CCCTGCCGCT TACCGGATAC |
| 951 | CTGTCCGCCT TTCTCCCTTC GGGAAGCGTG GCGCTTTCTC ATAGCTCACG |
| 1001 | CTGTAGGTAT CTCAGTTCGG TGTAGGTCGT TCGCTCCAAG CTGGGCTGTG |
| 1051 | TGCACGAACC CCCCGTTCAG CCCGACCGCT GCGCCTTATC CGGTAACTAT |
| 1101 | CGTCTTGAGT CCAACCCGGT AAGACACGAC TTATCGCCAC TGGCAGCAGC |
| 1151 | CACTGGTAAC AGGATTAGCA GAGCGAGGTA TGTAGGCGGT GCTACAGAGT |
| 1201 | TCTTGAAGTG GTGGCCTAAC TACGGCTACA CTAGAAGGAC AGTATTTGGT |
| 1251 | ATCTGCGCTC TGCTGAAGCC AGTTACCTTC GGAAAAAGAG TTGGTAGCTC |
| 1301 | TTGATCCGGC AAACAAACCA CCGCTGGTAG CGGTGGTTTT TTTGTTTGCA |
| 1351 | AGCAGCAGAT TACGCGCAGA AAAAAAGGAT CTCAAGAAGA TCCTTTGATC |
| 1401 | TTTTCTACGG GGTCTGACGC TCAGTGGAAC GAAAACTCAC GTTAAGGGAT |
| 1451 | TTTGGTCATG AGATTATCAA AAAGGATCTT CACCTAGATC CTTTTAAATT |
| 1501 | AAAAATGAAG TTTTAAATCA ATCTAAAGTA TATATGAGTA AACTTGGTCT |
| 1551 | GACAGTTACC AATGCTTAAT CAGTGAGGCA CCTATCTCAG CGATCTGTCT |
| 1601 | ATTTCGTTCA TCCATAGTTG CCTGACTCCC CGTCGTGTAG ATAACTACGA |
| 1651 | TACGGGAGGG CTTACCATCT GGCCCCAGTG CTGCAATGAT ACCGCGAGAC |
| 1701 | CCACGCTCAC CGGCTCCAGA TTTATCAGCA ATAAACCAGC CAGCCGGAAG |
| 1751 | GGCCGAGCGC AGAAGTGGTC CTGCAACTTT ATCCGCCTCC ATCCAGTCTA |
| 1801 | TTAATTGTTG CCGGGAAGCT AGAGTAAGTA GTTCGCCAGT TAATAGTTTG |
| 1851 | CGCAACGTTG TTGCCATTGC TGCAGGCATC GTGGTGTCAC GCTCGTCGTT |
| 1901 | TGGTATGGCT TCATTCAGCT CCGGTTCCCA ACGATCAAGG CGAGTTACAT |
| 1951 | GATCCCCCAT GTTGTGCAAA AAAGCGGTTA GCTCCTTCGG TCCTCCGATC |
| 2001 | GTTGTCAGAA GTAAGTTGGC CGCAGTGTTA TCACTCATGG TTATGGCAGC |
| 2051 | ACTGCATAAT TCTCTTACTG TCATGCCATC CGTAAGATGC TTTTCTGTGA |
| 2101 | CTGGTGAGTA CTCAACCAAG TCATTCTGAG AATAGTGTAT GCGGCGACCG |
| 2151 | AGTTGCTCTT GCCCGGCGTC AACACGGGAT AATACCGCGC CACATAGCAG |
| 2201 | AACTTTAAAA GTGCTCATCA TTGGAAAACG TTCTTCGGGG CGAAAACTCT |
| 2251 | CAAGGATCTT ACCGCTGTTG AGATCCAGTT CGATGTAACC CACTCGTGCA |
| 2301 | CCCAACTGAT CTTCAGCATC TTTTACTTTC ACCAGCGTTT CTGGGTGAGC |
| 2351 | AAAAACAGGA AGGCAAAATG CCGCAAAAAA GGGAATAAGG GCGACACGGA |
| 2401 | AATGTTGAAT ACTCATACTC TTCCTTTTTC AATATTATTG AAGCATTTAT |
| 2451 | CAGGGTTATT GTCTCATGAG CGGATACATA TTTGAATGTA TTTAGAAAAA |
| 2501 | TAAACAAATA GGGGTTCCGC GCACATTTCC CCGAAAAGTG CCACCTGACG |
| 2551 | TCTAAGAAAC CATTATTATC ATGACATTAA CCTATAAAAA TAGGCGTATC |
| 2601 | ACGAGGCCCT TTCGTCTTCA AGAATTCAGC TTGGCTGCAG TGAATAATAA |
| 2651 | AATGTGTGTT TGTCCGAAAT ACGCGTTTTG AGATTTCTGT CGCCGACTAA |
| 2701 | ATTCATGTCG CGCGATAGTG GTGTTTATCG CCGATAGAGA TGGCGATATT |
| 2751 | GGAAAAATCG ATATTTGAAA ATATGGCATA TTGAAAATGT CGCCGATGTG |
| 2801 | AGTTTCTGTG TAACTGATAT CGCCATTTTT CCAAAAGTGA TTTTTGGGCA |
| 2851 | TACGCGATAT CTGGCGATAG CGCTTATATC GTTTACGGGG GATGGCGATA |
| 2901 | GACGACTTTG GTGACTTGGG CGATTCTGTG TGTCGCAAAT ATCGCAGTTT |
| 2951 | CGATATAGGT GACAGACGAT ATGAGGCTAT ATCGCCGATA GAGGCGACAT |
| 3001 | CAAGCTGGCA CATGGCCAAT GCATATCGAT CTATACATTG AATCAATATT |
| 3051 | GGCCATTAGC CATATTATTC ATTGGTTATA TAGCATAAAT CAATATTGGC |
| 3101 | TATTGGCCAT TGCATACGTT GTATCCATAT CATAATATGT ACATTTATAT |
| 3151 | TGGCTCATGT CCAACATTAC CGCCATGTTG ACATTGATTA TTGACTAGTT |
| 3201 | ATTAATAGTA ATCAATTACG GGGTCATTAG TTCATAGCCC ATATATGGAG |
| 3251 | TTCCGCGTTA CATAACTTAC GGTAAATGGC CCGCCTGGCT GACCGCCCAA |
| 3301 | CGACCCCCGC CCATTGACGT CAATAATGAC GTATGTTCCC ATAGTAACGC |
| 3351 | CAATAGGGAC TTTCCATTGA CGTCAATGGG TGGAGTATTT ACGGTAAACT |
| 3401 | GCCCACTTGG CAGTACATCA AGTGTATCAT ATGCCAAGTA CGCCCCCTAT |
| 3451 | TGACGTCAAT GACGGTAAAT GGCCCGCCTG GCATTATGCC CAGTACATGA |
| 3501 | CCTTATGGGA CTTTCCTACT TGGCAGTACA TCTACGTATT AGTCATCGCT |
| 3551 | ATTACCATGG TGATGCGGTT TTGGCAGTAC ATCAATGGGC GTGGATAGCG |
| 3601 | GTTTGACTCA CGGGGATTTC CAAGTCTCCA CCCCATTGAC GTCAATGGGA |
| 3651 | GTTTGTTTTG GCACCAAAAT CAACGGGACT TTCCAAAATG TCGTAACAAC |
| 3701 | TCCGCCCCAT TGACGCAAAT GGGCGGTAGG CGTGTACGGT GGGAGGTCTA |
| 3751 | TATAAGCAGA GCTCGTTTAG TGAACCGTCA GATCGCCTGG AGACGCCATC |
| 3801 | CACGCTGTTT TGACCTCCAT AGAAGACACC GGGACCGATC CAGCCTCCGC |
| 3851 | AAGCTTGATA TCGAATTCCT GCAGCCCGGG GGATCCGCCC GCTTGCCGCC |
| 3901 | ACCATGGAGA CCCCCGCCCA GCTGCTGTTC CTGCTGCTGC TGTGGCTGCC |
| 3951 | CGACACCACC GGCGACATTC TGCTGACCCA GTCTCCAGCC ACCCTGTCTC |
| 4001 | TGAGTCCAGG AGAAAGAGCC ACTCTCTCCT GCAGGGCCAG TCAGAACATT |
| 4051 | GGCACAAGCA TACAGTGGTA TCAACAAAAA CCAGGTCAGG CTCCAAGGCT |
| 4101 | TCTCATAAGG TCTTCTTCTG AGTCTATCTC TGGGATCCCT TCCAGGTTTA |
| 4151 | GTGGCAGTGG ATCAGGGACA GATTTTACTC TTACCATCAG CAGTCTGGAG |
| 4201 | CCTGAAGATT TTGCAGTGTA TTACTGTCAA CAAAGTAATA CCTGGCCATT |
| 4251 | CACGTTCGGC CAGGGGACCA AGCTGGAGAT CAAACGTGAG TATTCTAGAA |
| 4301 | AGATCCTAGA ATTCTAAACT CTGAGGGGGT CGGATGACGT GGCCATTCTT |
| 4351 | TGCCTAAAGC ATTGACTTTA CTGCAAGGTC AGAAAAGCAT GCAAAGCCCT |
| 4401 | CAGAATGGCT GCAAAGAGCT CCAACAAAAC AATTTAGAAC TTTATTAAGG |
| 4451 | AATAGGGGGA AGCTAGGAAG AAACTCAAAA CATCAAGATT TTAAATACGC |
| 4501 | TTCTTGGTCT CCTTGCTATA ATTATCTGGG ATAAGCATGC TGTTTTCTGT |
| 4551 | CTGTCCCTAA CATGCCCTGT GATTATCCGC AAACAACACA CCCAAGGGCA |
| 4601 | GAACTTTGTT ACTTAAACAC CATCCTGTTT GCTTCTTTCC TCAGGAACTG |
| 4651 | TGGCTGCACC ATCTGTCTTC ATCTTCCCGC CATCTGATGA GCAGTTGAAA |
| 4701 | TCTGGAACTG CCTCTGTTGT GTGCCTGCTG AATAACTTCT ATCCCAGAGA |
| 4751 | GGCCAAAGTA CAGTGGAAGG TGGATAACGC CCTCCAATCG GGTAACTCCC |
| 4801 | AGGAGAGTGT CACAGAGCAG GACAGCAAGG ACAGCACCTA CAGCCTCAGC |
| 4851 | AGCACCCTGA CGCTGAGCAA AGCAGACTAC GAGAAACACA AAGTCTACGC |
| 4901 | CTGCGAAGTC ACCCATCAGG GCCTGAGCTC GCCCGTCACA AAGAGCTTCA |
| 4951 | ACAGGGGAGA GTGTTAGAGG GAGAAGTGCC CCCACCTGCT CCTCAGTTCC |
| 5001 | AGCCTGACCC CCTCCCATCC TTTGGCCTCT GACCCTTTTT CCACAGGGGA |
| 5051 | CCTACCCCTA TTGCGGTCCT CCAGCTCATC TTTCACCTCA CCCCCCTCCT |
| 5101 | CCTCCTTGGC TTTAATTATG CTAATGTTGG AGGAGAATGA ATAAATAAAG |
| 5151 | TGAATCTTTG CACCTGTGGT TTCTCTCTTT CCTCATTTAA TAATTATTAT |
| 5201 | CTGTTGTTTA CCAACTACTC AATTTCTCTT ATAAGGGACT AAATATGTAG |
| 5251 | TCATCCTAAG GCGCATAACC ATTTATAAAA ATCATCCTTC ATTCTATTTT |
| 5301 | ACCCTATCAT CCTCTGCAAG ACAGTCCTCC CTCAAACCCA CAAGCCTTCT |
| 5351 | GTCCTCACAG TCCCCTGGGC CATGGTAGGA GAGACTTGCT TCCTTGTTTT |
| 5401 | CCCCTCCTCA GCAAGCCCTC ATAGTCCTTT TTAAGGGTGA CAGGTCTTAC |
| 5451 | AGTCATATAT CCTTTGATTC AATTCCCTGA GAATCAACCA AAGCAAATTT |
| 5501 | TTCAAAAGAA GAAACCTGCT ATAAAGAGAA TCATTCATTG CAACATGATA |
| 5551 | TAAAATAACA ACACAATAAA AGCAATTAAA TAAACAAACA ATAGGGAAAT |
| 5601 | GTTTAAGTTC ATCATGGTAC TTAGACTTAA TGGAATGTCA TGCCTTATTT |
| 5651 | ACATTTTTAA ACAGGTACTG AGGGACTCCT GTCTGCCAAG GGCCGTATTG |
| 5701 | AGTACTTTCC ACAACCTAAT TTAATCCACA CTATACTGTG AGATTAAAAA |
| 5751 | CATTCATTAA AATGTTGCAA AGGTTCTATA AAGCTGAGAG ACAAATATAT |
| 5801 | TCTATAACTC AGCAATCCCA CTTCTAGATG ACTGAGTGTC CCCACCCACC |
| 5851 | AAAAAACTAT GCAAGAATGT TCAAAGCAGC TTTATTTACA AAAGCCAAAA |
| 5901 | ATTGGAAATA GCCCGATTGT CCAACAATAG AATGAGTTAT TAAACTGTGG |
| 5951 | TATGTTTATA CATTAGAATA CCCAATGAGG AGAATTAACA AGCTACAACT |
| 6001 | ATACCTACTC ACACAGATGA ATCTCATAAA AATAATGTTA CATAAGAGAA |
| 6051 | ACTCAATGCA AAAGATATGT TCTGTATGTT TTCATCCATA TAAAGTTCAA |
| 6101 | AACCAGGTAA AAATAAAGTT AGAAATTTGG ATGGAAATTA CTCTTAGCTG |
| 6151 | GGGGTGGGCG AGTTAGTGCC TGGGAGAAGA CAAGAAGGGG CTTCTGGGGT |
| 6201 | CTTGGTAATG TTCTGTTCCT CGTGTGGGGT TGTGCAGTTA TGATCTGTGC |
| 6251 | ACTGTTCTGT ATACACATTA TGCTTCAAAA TAACTTCACA TAAAGAACAT |
| 6301 | CTTATACCCA GTTAATAGAT AGAAGAGGAA TAAGTAATAG GTCAAGACCA |
| 6351 | CGCAGCTGGT AAGTGGGGGG GCCTGGGATC AAATAGCTAC CTGCCTAATC |
| 6401 | CTGCCCTCTT GAGCCCTGAA TGAGTCTGCC TTCCAGGGCT CAAGGTGCTC |
| 6451 | AACAAAACAA CAGGCCTGCT ATTTTCCTGG CATCTGTGCC CTGTTTGGCT |
| 6501 | AGCTAGGAGC ACACATACAT AGAAATTAAA TGAAACAGAC CTTCAGCAAG |
| 6551 | GGGACAGAGG ACAGAATTAA CCTTGCCCAG ACACTGGAAA CCCATGTATG |
| 6601 | AACACTCACA TGTTTGGGAA GQGGGAAGGG CACATGTAAA TGAGGACTCT |
| 6651 | TCCTCATTCT ATGGGGCACT CTGGCCCTGC CCCTCTCAGC TACTCATCCA |
| 6701 | TCCAACACAC CTTTCTAAGT ACCTCTCTCT GCCTACACTC TGAAGGGGTT |
| 6751 | CAGGAGTAAC TAACACAGCA TCCCTTCCCT CAAATGACTG ACAATCCCTT |
| 6801 | TGTCCTGCTT TGTTTTTCTT TCCAGTCAGT ACTGGGAAAG TGGGGAAGGA |
| 6851 | CAGTCATGGA GAAACTACAT AAGGAAGCAC CTTGCCCTTC TGCCTCTTGA |
| 6901 | GAATGTTGAT GAGTATCAAA TCTTTCAAAC TTTGGAGGTT TGAGTAGGGG |
| 6951 | TGAGACTCAG TAATGTCCCT TCCAATGACA TGAACTTGCT CACTCATCCC |
| 7001 | TGGGGGCCAA ATTGAACAAT CAAAGGCAGG CATAATCCAG CTATGAATTC |
| 7051 | TAGGATCGAT CCAGACATGA TAAGATACAT TGATGAGTTT GGACAAACCA |
| 7101 | CAACTAGAAT GCAGTGAAAA AAATGCTTTA TTTGTGAAAT TTGTGATGCT |
| 7151 | ATTGCTTTAT TTGTAACCAT TATAAGCTGC AATAAACAAG TTAACAACAA |
| 7201 | CAATTGCATT CAGTTTATGT TTCAGGTTCA GGGGGAGGTG TGGGAGGTTT |
| 7251 | TTTAAAGCAA GTAAAACCTC TACAAATGTG GTATGGCTGA TTATGATCTC |
| 7301 | TAGTCAAGGC ACTATACATC AAATATTCCT TATTAACCCC TTTACAAATT |
| 7351 | AAAAAGCTAA AGGTACACAA TTTTTGAGCA TAGTTATTAA TAGCAGACAC |
| 7401 | TCTATGCCTG TGTGGAGTAA GAAAAAACAG TATGTTATGA TTATAACTGT |
| 7451 | TATGCCTACT TATAAAGGTT ACAGAATATT TTTCCATAAT TTTCTTGTAT |
| 7501 | AGCAGTGCAG CTTTTTCCTT TGTGGTGTAA ATAGCAAAGC AAGCAAGAGT |
| 7551 | TCTATTACTA AACACAGCAT GACTCAAAAA ACTTAGCAAT TCTGAAGGAA |
| 7601 | AGTCCTTGGG GTCTTCTACC TTTCTCTTCT TTTTTGGAGG AGTAGAATGT |
| 7651 | TGAGAGTCAG CAGTAGCCTC ATCATCACTA GATGGCATTT GGTTCCTTCA |
| 7701 | AAACAGGTTT TCCTCATTAA AGGCATTCCA CCACTGCTCC CATTCATCAG |
| 7751 | TTCCATAGGT TGGAATCTAA AATACACAAA CAATTAGAAT CAGTAGTTTA |
| 7801 | ACACATTATA CACTTAAAAA TTTTATATTT ACCTTAGAGC TTTAAATCTC |
| 7851 | TGTAGGTAGT TTGTCCAATT ATGTCACACC ACAGAAGTAA GGTTCCTTCA |
| 7901 | CAAAGATCCG GGACCAAAGC GGCCATCGTG CCTCCCCACT CCTGCAGTTC |
| 7951 | GGGGGCATGG ATGCGCGGAT AGCCGCTGCT GGTTTCCTGG ATGCCGACGG |
| 8001 | ATTTGCACTG CCGGTAGAAC TCCGCGAGGT CGTCCAGCCT CAGGCAGCAG |
| 8051 | CTGAACCAAC TCGCGAGGGG ATCGAGCCCG GGGTGGGCGA AGAACTCCAG |
| 8101 | CATGAGATCC CCGCGCTGGA GGATCATCCA GCCGGCGTCC CGGAAAACGA |
| 8151 | TTCCGAAGCC CAACCTTTCA TAGAAGGCGG CGGTGGAATC GAAATCTCGT |
| 8201 | GATGGCAGGT TGGGCGTCGC TTGGTCGGTC ATTTCGAACC CCAGAGTCCC |
| 8251 | GCTCAGAAGA ACTCGTCAAG AAGGCGATAG AAGGCGATGC GCTGCGAATC |
| 8301 | GGGAGCGGCG ATACCGTAAA GCACGAGGAA GCGGTCAGCC CATTCGCCGC |
| 8351 | CAAGCTCTTC AGCAATATCA CGGGTAGCCA ACGCTATGTC CTGATAGCGG |
| 8401 | TCCGCCACAC CCAGCCGGCC ACAGTCGATG AATCCAGAAA AGCGGCCATT |
| 8451 | TTCCACCATG ATATTCGGCA AGCAGGCATC GCCATGGGTC ACGACGAGAT |
| 8501 | CCTCGCCGTC GGGCATGCGC GCCTTGAGCC TGGCGAACAG TTCGGCTGGC |
| 8551 | GCGAGCCCCT GATGCTCTTC GTCCAGATCA TCCTGATCGA CAAGACCGGC |
| 8601 | TTCCATCCGA GTACGTGCTC GCTCGATGCG ATGTTTCGCT TGGTGGTCGA |
| 8651 | ATGGGCAGGT AGCCGGATCA AGCGTATGCA GCCGCCGCAT TGCATCAGCC |
| 8701 | ATGATGGATA CTTTCTCGGC AGGAGCAAGG TGAGATGACA GGAGATCCTG |
| 8751 | CCCCGGCACT TCGCCCAATA GCAGCCAGTC CCTTCCCGCT TCAGTGACAA |
| 8801 | CGTCGAGCAC AGCTGCGCAA GGAACGCCCG TCGTGGCCAG CCACGATAGC |
| 8851 | CGCGCTGCCT CGTCCTGCAG TTCATTCAGG GCACCGGACA GGTCGGTCTT |
| 8901 | GACAAAAAGA ACCGGGCGCC CCTGCGCTGA CAGCCGGAAC ACGGCGGCAT |
| 8951 | CAGAGCAGCC GATTGTCTGT TGTGCCCAGT CATAGCCGAA TAGCCTCTCC |
| 9001 | ACCCAAGCGG CCGGAGAACC TGCGTGCAAT CCATCTTGTT CAATCATGCG |
| 9051 | AAACGATCCT CATCCTGTCT CTTGATCAGA TCTTGATCCC CTGCGCCATC |
| 9101 | AGATCCTTGG CGGCAAGAAA GCCATCCAGT TTACTTTGCA GGGCTTCCCA |
| 9151 | ACCTTACCAG AGGGCGCCCC AGCTGGCAAT TCCGGTTCGC TTGCTGTCCA |
| 9201 | TAAAACCGCC CAGTCTAGCT ATCGCCATGT AAGCCCACTG CAAGCTACCT |
| 9251 | GCTTTCTCTT TGCGCTTGCG TTTTCCCTTG TCCAGATAGC CCAGTAGCTG |
| 9301 | ACATTCATCC GGGGTCAGCA CCGTTTCTGC GGACTGGCTT TCTACGTGTT |
| 9351 | CCGCTTCCTT TAGCAGCCCT TGCGCCCTGA GTGCTTGCGG CAGCGTGAAG |

### SEQ ID NO:18

Nucleotide Sequence of the Expression Vector HCMV-K HuAb-VL1 hum V2 (Complete DNA Sequence of a Humanised Light Chain Expression Vector Comprising SEQ ID NO: 13 (humV2=VLm) from 3926-4246)

| | |
|---|---|
| 1 | CTAGCTTTTT GCAAAAGCCT AGGCCTCCAA AAAAGCCTCC TCACTACTTC |
| 51 | TGGAATAGCT CAGAGGCCGA GGCGGCCTCG GCCTCTGCAT AAATAAAAAA |
| 101 | AATTAGTCAG CCATGGGGCG GAGAATGGGC GGAACTGGGC GGAGTTAGGG |
| 151 | GCGGGATGGG CGGAGTTAGG GGCGGGACTA TGGTTGCTGA CTAATTGAGA |
| 201 | TGCATGCTTT GCATACTTCT GCCTGCTGGG GAGCCTGGTT GCTGACTAAT |
| 252 | TGAGATGCAT GCTTTGCATA CTTCTGCCTG CTGGGGAGCC TGGGGACTTT |
| 301 | CCACACCCTA ACTGACACAC ATTCCACAGC TGCCTCGCGC GTTTCGGTGA |
| 351 | TGACGGTGAA AACCTCTGAC ACATGCAGCT CCCGGAGACG GTCACAGCTT |
| 402 | GTCTGTAAGC GGATGCCGGG AGCAGACAAG CCCGTCAGGG CGCGTCAGCG |
| 451 | GGTGTTGGCG GGTGTCGGGG CGCAGCCATG ACCCAGTCAC GTAGCGATAG |
| 501 | CGGAGTGTAT ACTGGCTTAA CTATGCGGCA TCAGAGCAGA TTGTACTGAG |
| 551 | AGTGCACCAT ATGCGGTGTG AAATACCGCA CAGATGCGTA AGGAGAAAAT |
| 601 | ACCGCATCAG GCGCTCTTCC GCTTCCTCGC TCACTGACTC GCTGCGCTCG |
| 651 | GTCGTTCGGC TGCGGCGAGC GGTATCAGCT CACTCAAAGG CGGTAATACG |
| 702 | GTTATCCACA GAATCAGGGG ATAACGCAGG AAAGAACATG TGAGCAAAAG |
| 751 | GCCAGCAAAA GGCCAGGAAC CGTAAAAAGG CCGCGTTGCT GGCGTTTTTC |
| 801 | CATAGGCTCC GCCCCCCTGA CGAGCATCAC AAAAATCGAC GCTCAAGTCA |
| 851 | GAGGTGGCGA AACCCGACAG GACTATAAAG ATACCAGGCG TTTCCCCCTG |
| 901 | GAAGCTCCCT CGTGCGCTCT CCTGTTCCGA CCCTGCCGCT TACCGGATAC |
| 951 | CTGTCCGCCT TTCTCCCTTC GGGAAGCGTG GCGCTTTCTC ATAGCTCACG |
| 1001 | CTGTAGGTAT CTCAGTTCGG TGTAGGTCGT TCGCTCCAAG CTGGGCTGTG |
| 1051 | TGCACGAACC CCCCGTTCAG CCCGACCGCT GCGCCTTATC CGGTAACTAT |
| 1101 | CGTCTTGAGT CCAACCCGGT AAGACACGAC TTATCGCCAC TGGCAGCAGC |
| 1151 | CACTGGTAAC AGGATTAGCA GAGCGAGGTA TGTAGGCGGT GCTACAGAGT |
| 1201 | TCTTGAAGTG GTGGCCTAAC TACGGCTACA CTAGAAGGAC AGTATTTGGT |
| 1251 | ATCTGCGCTC TGCTGAAGCC AGTTACCTTC GGAAAAAGAG TTGGTAGCTC |
| 1301 | TTGATCCGGC AAACAAACCA CCGCTGGTAG CGGTGGTTTT TTTGTTTGCA |
| 1351 | AGCAGCAGAT TACGCGCAGA AAAAAAGGAT CTCAAGAAGA TCCTTTGATC |
| 1401 | TTTTCTACGG GGTCTGACGC TCAGTGGAAC GAAAACTCAC GTTAAGGGAT |
| 1451 | TTTGGTCATG AGATTATCAA AAAGGATCTT CACCTAGATC CTTTTAAATT |
| 1501 | AAAAATGAAG TTTTAAATCA ATCTAAAGTA TATATGAGTA AACTTGGTCT |
| 1551 | GACAGTTACC AATGCTTAAT CAGTGAGGCA CCTATCTCAG CGATCTGTCT |
| 1601 | ATTTCGTTCA TCCATAGTTG CCTGACTCCC CGTCGTGTAG ATAACTACGA |
| 1651 | TACGGGAGGG CTTACCATCT GGCCCCAGTG CTGCAATGAT ACCGCGAGAC |
| 1701 | CCACGCTCAC CGGCTCCAGA TTTATCAGCA ATAAACCAGC CAGCCGGAAG |
| 1751 | GGCCGAGCGC AGAAGTGGTC CTGCAACTTT ATCCGCCTCC ATCCAGTCTA |
| 1801 | TTAATTGTTG CCGGCAAGCT AGAGTAAGTA GTTCGCCAGT TAATAGTTTG |
| 1851 | CGCAACGTTQ TTGCCATTGC TGCAGGCATC GTGGTGTCAC GCTCGTCGTT |
| 1901 | TGGTATGGCT TCATTCAGCT CCGGTTCCCA ACGATCAAGG CGAGTTACAT |
| 1951 | GATCCCCCAT GTTGTGCAAA AAAGCGGTTA GCTCCTTCGG TCCTCCGATC |
| 2001 | GTTGTCAGAA GTAAGTTGGC CGCAGTGTTA TCACTCATGG TTATGGCAGC |
| 2051 | ACTGCATAAT TCTCTTACTG TCATGCCATC CGTAAGATGC TTTTCTGTGA |
| 2101 | CTGGTGAGTA CTCAACCAAG TCATTCTGAG AATAGTGTAT GCGGCGACCG |
| 2151 | AGTtGCTCTT GCCCGGCGTC AACACGGGAT AATACCGCGC CACATAGCAG |
| 2201 | AACTTTAAAA GTGCTCATCA TTGGAAAACG TTCTTCGGGG CGAAAACTCT |
| 2251 | CAAGGATCTT ACCGCTGTTG AGATCCAGTT CGATGTAACC CACTCGTGCA |
| 2301 | CCCAACTGAT CTTCAGCATC TTTTACTTTC ACCAGCGTTT CTGGGTGAGC |
| 2351 | AAAAACAGGA AGGCAAAATG CCGCAAAAAA GGGAATAAGG GCGACACGGA |
| 2401 | AATGTTGAAT ACTCATACTC TTCCTTTTTC AATATTATTG AAGCATTTAT |
| 2451 | CAGGGTTATT GTCTCATGAG CGGATACATA TTTGAATGTA TTTAGAAAAA |
| 2501 | TAAACAAATA GGGGTTCCGC GCACATTTCC CCGAAAAGTG CCACCTGACG |
| 2551 | TCTAAGAAAC CATTATTATC ATGACATTAA CCTATAAAAA TAGGCGTATC |
| 2601 | ACGAGGCCCT TTCGTCTTCA AGAATTCAGC TTGGCTGCAG TGAATAATAA |
| 2651 | AATGTGTGTT TGTCCGAAAT ACGCGTTTTG AGATTTCTGT CGCCGACTAA |
| 2701 | ATTCATGTCG CGCGATAGTG GTGTTTATCG CCGATAGAGA TGGCGATATT |
| 2751 | GGAAAAATCG ATATTTGAAA ATATGGCATA TTGAAAATGT CGCCGATGTG |
| 2801 | AGTTTCTGTG TAACTGATAT CGCCATTTTT CCAAAAGTGA TTTTTGGGCA |
| 2851 | TACGCGATAT CTGGCGATAG CGCTTATATC GTTTACGGGG GATGGCGATA |
| 2901 | GACGACTTTG GTGACTTGGG CQATTCTGTG TGTCGCAAAT ATCGCAGTTT |
| 2951 | CGATATAGGT GACAGACGAT ATGAGGCTAT ATCGCCGATA GAGGCGACAT |
| 3001 | CAAGCTGGCA CATGGCCAAT GCATATCGAT CTATACATTG AATCAATATT |
| 3051 | GGCCATTAGC CATATTATTC ATTGGTTATA TAGCATAAAT CAATATTGGC |
| 3101 | TATTGGCCAT TGCATACGTT GTATCCATAT CATAATATGT ACATTTATAT |
| 3151 | TGGCTCATGT CCAACATTAC CGCCATGTTG ACATTGATTA TTGACTAGTT |
| 3201 | ATTAATAGTA ATCAATTACG GGGTCATTAG TTCATAGCCC ATATATGGAG |
| 3251 | TTCCGCGTTA CATAACITAC GGTAAATGGC CCGCCTGGCT GACCGCCCAA |
| 3301 | CGACCCCCGC CCATTGACGT CAATAATGAC GTATGTTCCC ATAGTAACGC |
| 3351 | CAATAGGGAC TTTCCATTGA CGTCAATGGG TGGAGTATTT ACGGTAAACT |
| 3401 | GCCCACTTGG CAGTACATCA AGTGTATCAT ATGCCAAGTA CGCCCCCTAT |
| 3451 | TGACGTCAAT GACGGTAAAT GGCCCGCCTG GCATTATGCC CAGTACATGA |
| 3501 | CCTTATGGGA CTTTCCTACT TGGCAGTACA TCTACGTATT AGTCATCGCT |
| 3551 | ATTACCATGG TGATGCGGTT TTGGCAGTAC ATCAATGGGC GTGGATAGCG |
| 3601 | GTTTGACTCA CGGGGATTTC CAAGTCTCCA CCCCATTGAC GTCAATGGGA |
| 3651 | GTTTGTTTTG GCACCAAAAT CAACGGGACT TTCCAAAATG TCGTAACAAC |
| 3701 | TCCGCCCCAT TGACGCAAAT GGGCGGTAGG CGTGTACGGT GGGAGGTCTA |
| 3751 | TATAAGCAGA GCTCGTTTAG TGAACCGTCA GATCGCCTGG AGACGCCATC |
| 3801 | CACGCTGTTT TGACCTCCAT AGAAGACACC GGGACCGATC CAGCCTCCGC |
| 3851 | AAGCTTGCCG CCACCATGGA GACCCCCGCC CAGCTGCTGT TCCTGCTGCT |
| 3901 | GCTGTGGCTG CCCGACACCA CCGGCGACAT TCTGCTGACC CAGTCTCCAG |
| 3951 | CCACCCTGTC TCTGAGTCCA GGAGAAAGAG CCACTTTCTC CTGCAGGGCC |
| 4001 | AGTCAGAACA TTGGCACAAG CATACAGTGG TATCAACAAA AAACAAATGG |
| 4051 | TGCTCCAAGG CTTCTCATAA GGTCTTCTTC TGAGTCTATC TCTGGGATCC |
| 4101 | CTTCCAGGTT TAGTGGCAGT GGATCAGGGA CAGATTTTAC TCTTACCATC |
| 4151 | AGCAGTCTGG AGCCTGAAGA TTTTGCAGTG TATTACTGTC AACAAAGTAA |
| 4201 | TACCTGGCCA TTCACGTTCG GCCAGGGGAC CAAGCTGGAG ATCAAACGTG |
| 4251 | AGTATTCTAG AAAGATCCTA GAATTCTAAA CTCTGAGGGG GTCGGATGAC |
| 4301 | GTGGCCATTC TTTGCCTAAA GCATTGAGTT TACTGCAAGG TCAGAAAAGC |
| 4351 | ATGCAAAGCC CTCAGAATGG CTGCAAAGAG CTCCAACAAA ACAATTTAGA |
| 4403 | ACTTTATTAA GGAATAGGGG GAAGCTAGGA AGAAACTCAA AACATCAAGA |
| 4451 | TTTTAAATAC GCTTCTTGGT CTCCTTGCTA TAATTATCTG GGATAAGCAT |
| 4501 | GCTGTTTTCT GTCTGTCCCT AACATGCCCT GTGATTATCC GCAAACAACA |
| 4551 | CACCCAAGGG CAGAACTTTG TTACTTAAAC ACCATCCTGT TTGCTTCTTT |
| 4601 | CCTCAGGAAC TGTGGCTGCA CCATCTGTCT TCATCTTCCC GCCATCTGAT |
| 4651 | GAGCAGTTGA AATCTGGAAC TGCCTCTGTT GTGTGCCTGC TGAATAACTT |
| 4701 | CTATCCCAGA GAGGCCAAAG TACAGTGGAA GGTGGATAAC GCCCTCCAAT |
| 4751 | CGGGTAACTC CCAGGAGAGT GTCACAGAGC AGGACAGCAA GGACAGCACC |
| 4801 | TACAGCCTCA GCAGCACCCT GACGCTGAGC AAAGCAGACT ACGAGAAACA |
| 4851 | CAAAGTCTAC GCCTGCGAAG TCACCCATCA GGGCCTGAGC TCGCCCGTCA |
| 4901 | CAAAGAGCTT CAACAGGGGA GAGTGTTAGA GGGAGAAGTG CCCCCACCTG |
| 4951 | CTCCTCAGTT CCAGCCTGAC CCCCTCCCAT CCTTTGGCCT CTGACCCTTT |
| 5001 | TTCCACAGGG GACCTACCCC TATTGCGGTC CTCCAGCTCA TCTTTCACCT |
| 5051 | CACCCCCCTC CTCCTCCTTG GCTTTAATTA TGCTAATGTT GGAGGAGAAT |
| 5101 | GAATAAATAA AGTGAATCTT TGCACCTGTG GTTTCTCTCT TTCCTCATTT |
| 5151 | AATAATTATT ATCTGTTGTT TACCAACTAC TCAATTTCTC TTATAAGGGA |
| 5201 | CTAAATATGT AGTCATCCTA AGGCGCATAA CCATTTATAA AAATCATCCT |
| 5251 | TCATTCTATT TTACCCTATC ATCCTCTGCA AGACAGTCCT CCCTCAAACC |
| 5301 | CACAAGCCTT CTGTCCTCAC AGTCCCCTGG GCCATGGTAG GAGAGACTTG |
| 5351 | CTTCCTTGTT TTCCCCTCCT CAGCAAGCCC TCATAGTCCT TTTTAAGGGT |
| 5401 | GACAGGTCTT ACAGTCATAT ATCCTTTGAT TCAATTCCCT GAGAATCAAC |
| 5451 | CAAAGCAAAT TTTTCAAAAG AAGAAACCTG CTATAAAGAG AATCATTCAT |
| 5501 | TGCAACATGA TATAAAATAA CAACACAATA AAAGCAATTA AATAAACAAA |
| 5551 | CAATAGGGAA ATGTTTAAGT TCATCATGGT ACTTAGACTT AATGGAATGT |
| 5601 | CATGCCTTAT TTACATTTTT AAACAGGTAC TGAGGGACTC CTGTCTGCCA |
| 5651 | AGGGCCGTAT TGAGTACTTT CCACAACCTA ATTTAATCCA CACTATACTG |
| 5701 | TGAGATTAAA AACATTCATT AAAATGTTGC AAAGGTTCTA TAAAGCTGAG |
| 5751 | AGACAAATAT ATTCTATAAC TCAGCAATCC CACTTCTAGA TGACTGAGTG |
| 5801 | TCCCCACCCA CCAAAAAACT ATGCAAGAAT GTTCAAAGCA GCTTTATTTA |
| 5851 | CAAAAGCCAA AAATTGGAAA TAGCCCGATT GTCCAACAAT AGAATGAGTT |
| 5901 | ATTAAACTGT GGTATGTTTA TACATTAGAA TACCCAATGA GGAGAATTAA |
| 5951 | CAAGCTACAA CTATACCTAC TCACACAGAT GAATCTCATA AAAATAATGT |
| 6001 | TACATAAGAG AAACTCAATG CAAAAGATAT GTTCTGTATG TTTTCATCCA |
| 6051 | TATAAAGTTC AAAACCAGGT AAAAATAAAG TTAGAAATTT GGATGGAAAT |
| 6101 | TACTCTTAGC TGGGGGTGGG CGAGTTAGTG CCTGGGAGAA GACAAGAAGG |
| 6151 | GGCTTCTGGG GTCTTGGTAA TGTTCTGTTC CTCGTGTGGG GTTGTGCAGT |
| 6201 | TATGATCTGT GCACTGTTCT GTATACACAT TATGCTTCAA AATAACTTCA |
| 6251 | CATAAAGAAC ATCTTATACC CAGTTAATAG ATAGAAGAGG AATAAGTAAT |
| 6301 | AGGTCAAGAC CACGCAGCTG GTAAGTGGGG GGGCCTGGGA TCAAATAGCT |
| 6351 | ACCTGCCTAA TCCTGCCCTC TTGAGCCCTG AATGAGTCTG CCTTCCAGGG |
| 6401 | CTCAAGGTGC TCAACAAAAC AACAGGCCTG CTATTTTCCT GGCATCTGTG |
| 6451 | CCCTGTTTGG CTAGCtAGGA GCACACATAC ATAGAAATTA AATGAAACAG |
| 6501 | ACCTTCAGCA AGGGGACAGA GGACAGAATT AACCTTGCCC AGACACTGGA |
| 6551 | AACCCATGTA TGAACACTCA CATGTTTGGG AAGGGGGAAG GGCACATGTA |
| 6601 | AATGAGGACT CTTCCTCATT CTATGGGGCA CTCTGGCCCT GCCCCTCTCA |
| 6651 | GCTACTCATC CATCCAACAC ACCTTTCTAA GTACCTCTCT CTGCCTACAC |
| 6701 | TCTGAAGGGG TTCAGGAGTA ACTAACACAG CATCCCTTCC CTCAAATGAC |
| 6751 | TGACAATCCC TTTGTCCTGC TTTGTTTTTC TTTCCAGTCA GTACTGGGAA |
| 6801 | AGTGGGGAAG GACAGTCATG GAQAAACTAC ATAAGGAAGC ACCTTGCCCT |
| 6851 | TCTGCCTCTT GAGAATGTTG ATGAGTATCA AATCTTTCAA ACTTTGGAGG |
| 6901 | TTTGAGTAGG GGTGAGACTC AGTAATGTCC CTTCCAATGA CATGAACTTG |
| 6951 | CTCACTCATC CCTGGGGGCC AAATTGAACA ATCAAAGGCA GGCATAATCC |
| 7001 | AGCTATGAAT TCTAGGATCG ATCCAGACAT GATAAGATAC ATTGATGAGT |
| 7051 | TTGGACAAAC CACAACTAGA ATGCAGTGAA AAAAATGCTT TATTTGTGAA |
| 7101 | ATTTGTGATG CTATTGCTTT ATTTGTAACC ATTATAAGCT GCAATAAACA |
| 7151 | AGTTAACAAC AACAATTGCA TTCATTTTAT GTTTCAGGTT CAGGGGGAGG |
| 7201 | TGTGGGAGGT TTTTTAAAGC AAGTAAAACC TCTACAAATG TGGTATGGCT |
| 7251 | GATTATGATC TCTAGTCAAG GCACTATACA TCAAATATTC CTTATTAACC |
| 7301 | CCTTTACAAA TTAAAAAGCT AAAGGTACAC AATTTTTGAG CATAGTTATT |
| 7351 | AATAGCAGAC ACTCTATGCC TGTGTGGAGT AAGAAAAAAC AGTATGTTAT |
| 7401 | GATTATAACT GTTATGCCTA CTTATAAAGG TTACAGAATA TTTTTCCATA |
| 7451 | ATTTTCTTGT ATAGCAGTGC AGCTTTTTCC TTTGTGGTGT AAATAGCAAA |
| 7501 | GCAAGCAAGA GTTCTATTAC TAAACACAGC ATGACTCAAA AAACTTAGCA |
| 7551 | ATTCTGAAGG AAAGTCCTTG GGGTCTTCTA CCTTTCTCTT CTTTTTTGGA |
| 7601 | GGAGTAGAAT GTTGAGAGTC AGCAGTAGCC TCATCATCAC TAGATGGCAT |
| 7651 | TTCTTCTGAG CAAAACAGGT TTTCCTCATT AAAGGCATTC CACCACTGCT |
| 7701 | CCCATTCATC AGTTCCATAG GTTGGAATCT AAAATACACA AACAATTAGA |
| 7751 | ATCAGTAGTT TAACACATTA TACACTTAAA AATTTTATAT TTACCTTAGA |
| 7801 | GCTTTAAATC TCTGTAGGTA GTTTGTCCAA TTATGTCACA CCACAGAAGT |
| 7851 | AAGGTTCCTT CACAAAGATC CGGGACCAAA GCGGCCATCG TGCCTCCCCA |
| 7901 | CTCCTGCAGT TCGGGGGCAT GGATGCGCGG ATAGCCGCTG CTGGTTTCCT |
| 7951 | GGATGCCGAC GGATTTGCAC TGCCGGTAGA ACTCCGCGAG GTCGTCCAGC |
| 8001 | CTCAGGCAGC AGCTGAACCA ACTCGCGAGG GGATCGAGCC CGGGGTGGGC |
| 8051 | GAAGAACTCC AGCATGAGAT CCCCGCGCTG GAGGATCATC CAGCCGGCGT |
| 8101 | CCCGGAAAAC GATTCCGAAG CCCAACCTTT CATAGAAGGC GGCGGTGGAA |
| 8151 | TCGAAATCTC GTGATGGCAG GTTGGGCGTC GCTTGGTCGG TCATTTCGAA |
| 8201 | CCCCAGAGTC CCGCTCAGAA GAACTCGTCA AGAAGGCGAT AGAAGGCGAT |
| 8251 | GCGCTGCGAA TCGGGAGCGG CGATACCGTA AAGCACGAGG AAGCGGTCAG |
| 8301 | CCCATTCGCC GCCAAGCTCT TCAGCAATAT CACGGGTAGC CAACGCTATG |
| 8351 | TCCTGATAGC GGTCCGCCAC ACCCAGCCGG CCACAGTCGA TGAATCCAGA |
| 8401 | AAAGCGGCCA TTTTCCACCA TGATATTCGG CAAGCAGGCA TCGCCATGGG |
| 8451 | TCACGACGAG ATCCTCGCCG TCGGGCATGC GCGCCTTGAG CCTGGCGAAC |
| 8501 | AGTTCGGCTG GCGCGAGCCC CTGATGCTCT TCGTCCAGAT CATCCTGATC |
| 8551 | GACAAGACCG GCTTCCATCC GAGTACGTGC TCGCTCGATG CGATGTTTCG |
| 8601 | CTTGGTGGTC GAATGGGCAG GTAGCCGGAT CAAGCGTATG CAGCCGCCGC |
| 8651 | ATTGCATCAG CCATGATGGA TACTTTCTCG GCAGGAGCAA GGTGAGATGA |
| 8701 | CAGGAGATCC TGCCCCGGCA CTTCGCCCAA TAGCAGCCAG TCCCTTCCCG |
| 8751 | CTTCAGTGAC AACGTCGAGC ACAGCTGCGC AAGGAACGCC CGTCGTGGCC |
| 8801 | AGCCACGATA GCCGCGCTCC CTCGTCCTGC AGTTCATTCA GGGCACCGGA |
| 8851 | CAGGTCGGTC TTGACAAAAA GAACCGGGCG CCCCTGCGCT GACAGCCGGA |
| 8901 | ACACGGCGGC ATCAGAGCAG CCGATTGTCT GTTGTGCCCA GTCATAGCCG |
| 8951 | AATAGCCTCT CCACCCAAGC GGCCGGAGAA CCTGCGTGCA ATCCATCTTG |
| 9001 | TTCAATCATG CGAAACGATC CTCATCCTGT CTCTTGATCA GATCTTGATC |
| 9051 | CCCTGCGCCA TCAGATCCTT GGCGGCAAGA AAGCCATCCA GTTTACTTTG |
| 9101 | CAGGGCTTCC CAACCTTACC AGAGGGCGCC CCAGCTGGCA ATTCCGGTTC |
| 9151 | GCTTGCTGTC CATAAAACCG CCCAGTCTAG CTATCGCCAT GTAAGCCCAC |
| 9201 | TGCAAGCTAC CTGCTTTCTC TTTGCGCTTG CGTTTTCCCT TGTCCAGATA |
| 9251 | GCCCAGTAGC TGACATTCAT CCGGGGTCAG CACCGTTTCT GCGGACTGGC |
| 9301 | TTTCTACGTG TTCCGCTTCC TTTAGCAGCC CTTGCGCCCT GAGTGCTTGC |
| 9351 | GGCAGCGTGA AG |

1. A binding molecule comprising at least one antigen binding site, comprising in sequence the hypervariable regions CDR1, CDR2 and CDR3, said CDR1 having the amino acid sequence Asn-Tyr-Ile-Ile-His (NYIIH), said CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) and said CDR3 having the amino acid sequence Ser-Gly-Pro-Tyr-Ala-Trp-Phe-Asp-Thr (SGPYAWFDT).

2. A binding molecule according to claim 1 comprising
a) a first domain comprising in sequence the hypervariable regions CDR1, CDR2 and CDR3, said CDR1 having the amino acid sequence Asn-Tyr-Ile-Ile-His (NYIIH), said CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) and said CDR3 having the amino acid sequence Ser-Gly-Pro-Tyr-Ala-Trp-Phe-Asp-Thr (SGPYAWFDT); and
b) a second domain comprising in sequence the hypervariable regions CDR1′, CDR2′ and CDR3′, CDR1¹′ having the amino acid sequence Arg-Ala-Ser-Gln-Asn-Ile-Gly-Thr-Ser-Ile-Gln (RASQNIGTSIQ), CDR2′ having the amino acid sequence Ser-Ser-Ser-Glu-Ser-Ile-Ser (SSSESIS) and CDR3′ having the amino acid sequence Gln-Gln-Ser-Asn-Thr-Trp-Pro-Phe-Thr (QQSNTWPFT).

3. A binding molecule according to any one of claims 1 or 2, which is a chimeric or humanised monoclonal antibody.

4. A binding molecule according to any one of claims 1 or 2, comprising a polypeptide of SEQ ID NO:1 and/or a polypeptide of SEQ ID NO:2.

5. A binding molecule according to any one of claims 1 or 2, comprising a polypeptide of SEQ ID NO:3 and/or a polypeptide of SEQ ID NO:4.

6. A binding molecule according to any one of claims 4 or 5 which is a chimeric monoclonal antibody.

7. A binding molecule which is a humanised antibody comprising a polypeptide of SEQ ID NO:9 or of SEQ ID NO:10 and a polypeptide of SEQ ID NO:7 or of SEQ ID NO:8.

8. A binding molecule which is a humanised antibody comprising
a polypeptide of SEQ ID NO:9 and a polypeptide of SEQ ID NO:7,
a polypeptide of SEQ ID NO:9 and a polypeptide of SEQ ID NO:8,
a polypeptide of SEQ ID NO:10 and a polypeptide of SEQ ID NO:7, or
a polypeptide of SEQ ID NO:10 and a polypeptide of SEQ ID NO:8.

9. Isolated polynucleotides comprising polynucleotides encoding a binding molecule according to any one of claims 1 to 8.

10. Polynucleotides according to claim 9 encoding the amino acid sequence of CDR1, CDR2 and CDR3 according to claim 2 and/or polynucleotides encoding the amino acid sequence of CDR1′, CDR2′ and CDR3′ according to claim 2.

11. Polynucleotides comprising a polynucleotide of SEQ ID NO: 5 and/or a polynucleotide of SEQ ID NO: 6.

12. Polynucletides comprising polynucleotides encoding a polypeptide of SEQ ID NO:7 or SEQ ID NO:8 and a polypeptide of SEQ ID NO:9 or SEQ ID NO:10.

13. Polynucleotides comprising a polynucleotide of SEQ ID NO:11 or of SEQ ID NO:12 and a polynucleotide of SEQ ID NO:13 or a polynucleotide of SEQ ID NO:14.

14. An expression vector comprising polynucleotides according to any one of claims 9 to 13.

15. An expression system comprising a polynucleotide according to any one of claims 9 to 13, wherein said expression system or part thereof is capable of producing a polypeptide of any one of claims 1 to 8, when said expression system or part thereof is present in a compatible host cell.

16. An isolated host cell which comprises an expression system according to claim 15.

17. Use of a molecule or of a humanised antibody according to any on of claims 1 to 8 as a pharmaceutical.

18. Use according to claim 17 in the treatment and/or prophylaxis of autoimmune diseases, transplant rejection, psoriasis, inflammatory bowel disease and allergies.

19. A pharmaceutical composition comprising a molecule or a humanised antibody according to any one of claims 1 to 8 in association with at least one pharmaceutically acceptable carrier or diluent.

20. A method of treatment and/or prophylaxis of diseases associated with autoimmune diseases, transplant rejection, psoriasis, inflammatory bowel disease and allergies comprising administering to a subject in need of such treatment and/or prophylaxis an effective amount of a molecule or a humanised antibody according to any one of claims 1 to 8.

21. Use of a binding molecule having a binding specificity for both CD45RO and CD45RB in medicine.

22. The use according to claim 21, wherein the binding molecule is a chimeric, a humanised or a fully human monoclonal antibody.

23. The use according to claim 21 or 22, wherein the binding molecule binds to a CD45RO isoform with a dissociation constant (Kd)<15 nM.

24. The use according to any of claims 21 to 23, wherein the binding molecule binds to a CD45RB isoform with a dissociation constant (Kd)<15 nM.

25. The use according to any of claims 21 to 24, wherein the binding molecule binds CD45 isoforms which
include the A and B epitopes, but not the C epitope of the CD45 molecule; and/or
include the B epitope, but not the A and not the C epitope of the CD45 molecule; and/or
isoforms which do not include any one of the A, B or C epitopes of the CD45 molecule.

26. The use according to any of claims 21 to 25, wherein the binding molecule does not bind CD45 isoforms which
include all of the A, B and C epitopes of the CD45 molecule; and/or
include both the B and C epitopes, but not the A epitope of the CD45 molecule.

27. The use according to any of claims 21 to 26, wherein the binding molecule binds to its target epitope on PEER cells, and wherein said binding is with a Kd<l5 nM.


SEQUENCE LISTING (30 sequences)

SEQ ID NO: 1 - protein, 107 aa, Artificial
  MISC_FEATURE: Part of the amino acid sequence of chimeric light chain
DILLTQSPAILSVSPGERVSFSCRASQNIGTSIQWYQQRTNGSPRLLIRSSSESISGIPSRFSGSGSGTDFTLSINSVESEDIADYYCQQSNTWPFTFGSGTKLEIK

SEQ ID NO: 2 - protein, 118 aa, Artificial
  MISC_FEATURE: Part of the amino acid sequence of chimeric heavy chain
EVQLQQSGPELVKPGASVKMSCKASGYTFTNYIIHWVKQEPGQGLEWIGYFNPYNHGTKYNEKFKGRATLTADKSSNTAYMDLSSLTSEDSAIYYCARSGPYAWFDTWGQGTTVTVSS

SEQ ID NO: 3 - protein, 214 aa, Artificial
  MISC_FEATURE: Amino acid sequence of chimeric light chain
DILLTQSPAILSVSPGERVSFSCRASQNIGTSIQWYQQRTNGSPRLLIRSSSESISGIPSRFSGSGSGTDFTLSINSVESEDIADYYCQQSNTWPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 448 aa, Artificial
  MISC_FEATURE: Amino acid sequence of chimeric heavy chain
EVQLQQSGPELVKPGASVKMSCKASGYTFTNYIIHWVKQEPGQGLEWIGYFNPYNHGTKYNEKFKGRATLTADKSSNTAYMDLSSLTSEDSAIYYCARSGPYAWFDTWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 321 bp, Artificial
  misc_feature: Nucleotide sequence encoding a polypeptide of SEQ ID NO1
gacattctgctgacccagtctccagccatcctgtctgtgagtccaggagaaagagtcagtttctcctgcagggccagtcagaacattggcacaagcatacagtggtatcaacaaagaacaaatggttctccaaggcttctcataaggtcttcttctgagtctatctctgggatcccttccaggtttagtggcagtggatcagggacagattttactcttagcatcaacagtgtggagtctgaagatattgcagattattactgtcaacaaagtaatacctggccattcacgttcggctcggggaccaagcttgaaatcaaa

SEQ ID NO: 6 - DNA, 354 bp, Artificial
  misc_feature: Nucleotide sequence encoding a polypeptide of SEQ ID NO2
gaggtgcagctgcagcagtcaggacctgaactggtaaagcctggggcttcagtgaagatgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcagacaaatcctccaacacagcctacatggacctcagcagcctgacctctgaggactctgcgatctactactgtgcaagatcaggaccctatgcctggtttgacacctggggccaagggaccacggtcaccgtctcctca

SEQ ID NO: 7 - protein, 107 aa, Artificial
  MISC_FEATURE: Part of amino acid sequence of humanised light chain designated humV2 (humV2 = VLm)
DILLTQSPATLSLSPGERATFSCRASQNIGTSIQWYQQKTNGAPRLLIRSSSESISGIPSRFSGSGSGTDFTLTISSLEPEDFAVYYCQQSNTWPFTFGQGTKLEIK

SEQ ID NO: 8 - protein, 107 aa, Artificial
  MISC_FEATURE: Part of amino acid sequence of humanised light chain designated humV1 (humV1 = VLh)
DILLTQSPATLSLSPGERATLSCRASQNIGTSIQWYQQKPGQAPRLLIRSSSESISGIPSRFSGSGSGTDFTLTISSLEPEDFAVYYCQQSNTWPFTFGQGTKLEIK

SEQ ID NO: 9 - protein, 118 aa, Artificial
  MISC_FEATURE: Part of amino acid sequence of humanised heavy chain designated VHE
EVQLVESGAEVKKPGASVKVSCKASGYTFTNYIIHWVKQEPGQGLEWIGYFNPYNHGTKYNEKFKGRATLTANKSISTAYMELSSLRSEDTAVYYCARSGPYAWFDTWGQGTTVTVSS

SEQ ID NO: 10 - protein, 118 aa, Artificial
  MISC_FEATURE: Part of amino acid sequence of humanised heavy chain designated VHQ
QVQLVESGAEVKKPGASVKVSCKASGYTFTNYIIHWVKQEPGQGLEWIGYFNPYNHGTKYNEKFKGRATLTANKSISTAYMELSSLRSEDTAVYYCARSGPYAWFDTWGQGTTVTVSS

SEQ ID NO: 11 - DNA, 354 bp, Artificial
  misc_feature: Nucleotide sequence encoding amino acid sequence SEQ ID NO9
gaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcaaacaaatccatcagcacagcctacatggagctcagcagcctgcgctctgaggacactgcggtctactactgtgcaagatcaggaccctatgcctggtttgacacctggggccaagggaccacggtcaccgtctcctca

SEQ ID NO: 12 - DNA, 354 bp, Artificial
  misc_feature: Nucleotide sequence encoding amino acid sequence SEQ ID NO10
caggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcaaacaaatccatcagcacagcctacatggagctcagcagcctgcgctctgaggacactgcggtctactactgtgcaagatcaggaccctatgcctggtttgacacctggggccaagggaccacggtcaccgtctcctca

SEQ ID NO: 13 - DNA, 321 bp, Artificial
  misc_feature: Nucleotide sequence encoding amino acid sequence SEQ ID NO7
gacattctgctgacccagtctccagccaccctgtctctgagtccaggagaaagagccactttctcctgcagggccagtcagaacattggcacaagcatacagtggtatcaacaaaaaacaaatggtgctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagctggagatcaaa

SEQ ID NO: 14 - DNA, 321 bp, Artificial
  misc_feature: Nucleotide sequence encoding amino acid sequence SEQ ID NO8
gacattctgctgacccagtctccagccaccctgtctctgagtccaggagaaagagccactctctcctgcagggccagtcagaacattggcacaagcatacagtggtatcaacaaaaaccaggtcaggctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagctggagatcaaa

SEQ ID NO: 15 - DNA, 8687 bp, Artificial
  misc_feature: Nucleotide sequence of the expression vector HCMV-G1 HuAb-VHQ (Complete DNA Sequence of a humanised heavy chain expression vect or comprising SEQ ID NO12 (VHQ) from 3921-4274)
agctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagcttggctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcaagcttgccgccaccatggactggacctggagggtgttctgcctgctggccgtggcccccggcgcccacagccaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcaaacaaatccatcagcacagcctacatggagctcagcagcctgcgctctgaggacactgcggtctactactgtgcaagatcaggaccctatgcctggtttgacacctggggccaagggaccacggtcaccgtctcctcaggtgagttctagaaggatcccaagctagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagatctgatatcatcgatgaattcgagctcggtacccggggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagct

SEQ ID NO: 16 - DNA, 8687 bp, Artificial
  misc_feature: Nucleotide sequence of the expression vector HCMV-G1 HuAb-VHE (Complete DNA Sequence of a humanised heavy chain expression vector comprising SEQ ID NO 11 (VHE) from 3921-4274)
agctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagcttggctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcaagcttgccgccaccatggactggacctggagggtgttctgcctgctggccgtggcccccggcgcccacagcgaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcaaacaaatccatcagcacagcctacatggagctcagcagcctgcgctctgaggacactgcggtctactactgtgcaagatcaggaccctatgcctggtttgacacctggggccaagggaccacggtcaccgtctcctcaggtgagttctagaaggatcccaagctagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagatctgatatcatcgatgaattcgagctcggtacccggggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagct

SEQ ID NO: 17 - DNA, 9400 bp, Artificial
  misc_feature: Nucleotide sequence of the expression vector HCMV-K HuAb-VL1 humV1 (Complete DNA Sequence of a humanised light chain expression vector comprising SEQ ID NO 14 (humV1=VLh) from 3964-4284
ctagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagcttggctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcaagcttgatatcgaattcctgcagcccgggggatccgcccgcttgccgccaccatggagacccccgcccagctgctgttcctgctgctgctgtggctgcccgacaccaccggcgacattctgctgacccagtctccagccaccctgtctctgagtccaggagaaagagccactctctcctgcagggccagtcagaacattggcacaagcatacagtggtatcaacaaaaaccaggtcaggctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagctggagatcaaacgtgagtattctagaaagatcctagaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgtttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctagatgactgagtgtccccacccaccaaaaaactatgcaagaatgttcaaagcagctttatttacaaaagccaaaaattggaaatagcccgattgtccaacaatagaatgagttattaaactgtggtatgtttatacattagaatacccaatgaggagaattaacaagctacaactatacctactcacacagatgaatctcataaaaataatgttacataagagaaactcaatgcaaaagatatgttctgtatgttttcatccatataaagttcaaaaccaggtaaaaataaagttagaaatttggatggaaattactcttagctgggggtgggcgagttagtgcctgggagaagacaagaaggggcttctggggtcttggtaatgttctgttcctcgtgtggggttgtgcagttatgatctgtgcactgttctgtatacacattatgcttcaaaataacttcacataaagaacatcttatacccagttaatagatagaagaggaataagtaataggtcaagaccacgcagctggtaagtgggggggcctgggatcaaatagctacctgcctaatcctgccctcttgagccctgaatgagtctgccttccagggctcaaggtgctcaacaaaacaacaggcctgctattttcctggcatctgtgccctgtttggctagctaggagcacacatacatagaaattaaatgaaacagaccttcagcaaggggacagaggacagaattaaccttgcccagacactggaaacccatgtatgaacactcacatgtttgggaagggggaagggcacatgtaaatgaggactcttcctcattctatggggcactctggccctgcccctctcagctactcatccatccaacacacctttctaagtacctctctctgcctacactctgaaggggttcaggagtaactaacacagcatcccttccctcaaatgactgacaatccctttgtcctgctttgtttttctttccagtcagtactgggaaagtggggaaggacagtcatggagaaactacataaggaagcaccttgcccttctgcctcttgagaatgttgatgagtatcaaatctttcaaactttggaggtttgagtaggggtgagactcagtaatgtcccttccaatgacatgaacttgctcactcatccctgggggccaaattgaacaatcaaaggcaggcataatccagctatgaattctaggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaag

SEQ ID NO: 18 - DNA, 9362 bp, Artificial
  misc_feature: Nucleotide sequence of the expression vector HCMV-K HuAb-VL1 humV2 (Complete DNA Sequence of a humanised light chain expression vector comprising SEQ ID NO 13 (humV2=VLm) from 3926-4246)
ctagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagcttggctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcaagcttgccgccaccatggagacccccgcccagctgctgttcctgctgctgctgtggctgcccgacaccaccggcgacattctgctgacccagtctccagccaccctgtctctgagtccaggagaaagagccactttctcctgcagggccagtcagaacattggcacaagcatacagtggtatcaacaaaaaacaaatggtgctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagctggagatcaaacgtgagtattctagaaagatcctagaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgtttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctagatgactgagtgtccccacccaccaaaaaactatgcaagaatgttcaaagcagctttatttacaaaagccaaaaattggaaatagcccgattgtccaacaatagaatgagttattaaactgtggtatgtttatacattagaatacccaatgaggagaattaacaagctacaactatacctactcacacagatgaatctcataaaaataatgttacataagagaaactcaatgcaaaagatatgttctgtatgttttcatccatataaagttcaaaaccaggtaaaaataaagttagaaatttggatggaaattactcttagctgggggtgggcgagttagtgcctgggagaagacaagaaggggcttctggggtcttggtaatgttctgttcctcgtgtggggttgtgcagttatgatctgtgcactgttctgtatacacattatgcttcaaaataacttcacataaagaacatcttatacccagttaatagatagaagaggaataagtaataggtcaagaccacgcagctggtaagtgggggggcctgggatcaaatagctacctgcctaatcctgccctcttgagccctgaatgagtctgccttccagggctcaaggtgctcaacaaaacaacaggcctgctattttcctggcatctgtgccctgtttggctagctaggagcacacatacatagaaattaaatgaaacagaccttcagcaaggggacagaggacagaattaaccttgcccagacactggaaacccatgtatgaacactcacatgtttgggaagggggaagggcacatgtaaatgaggactcttcctcattctatggggcactctggccctgcccctctcagctactcatccatccaacacacctttctaagtacctctctctgcctacactctgaaggggttcaggagtaactaacacagcatcccttccctcaaatgactgacaatccctttgtcctgctttgtttttctttccagtcagtactgggaaagtggggaaggacagtcatggagaaactacataaggaagcaccttgcccttctgcctcttgagaatgttgatgagtatcaaatctttcaaactttggaggtttgagtaggggtgagactcagtaatgtcccttccaatgacatgaacttgctcactcatccctgggggccaaattgaacaatcaaaggcaggcataatccagctatgaattctaggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaag

SEQ ID NO: 19 - protein, 11 aa, Artificial
  MISC_FEATURE: hypervariable regions CDR1′ in a CD45RO/RB binding molecule of SEQ ID NO1
RASQNIGTSIQ

SEQ ID NO: 20 - protein, 7 aa, Artificial
  MISC_FEATURE: hypervariable region CDR2′ in a CD45RO/RB binding molecule of SEQ ID NO1
SSSESIS

SEQ ID NO: 21 - protein, 9 aa, Artificial
  MISC_FEATURE: hypervariable region CDR3′ in a CD45RO/RB binding molecule of SEQ ID NO1
QQSNTWPFT

SEQ ID NO: 22 - protein, 5 aa, Artificial
  MISC_FEATURE: hypervariable region CDR1 in a CD45RO/RB binding molecule of SEQ ID NO2
NYIIH

SEQ ID NO: 23 - protein, 17 aa, Artificial
  MISC_FEATURE: hypervariable region CDR2 in a CD45RO/RB binding molecule of SEQ ID NO2
YFNPYNHGTKYNEKFKG

SEQ ID NO: 24 - protein, 9 aa, Artificial
  MISC_FEATURE: hypervariable region CDR3 in a CD45RO/RB binding molecule of SEQ ID NO2
SGPYAWFDT

SEQ ID NO: 25 - DNA, 33 bp, Artificial
  misc_feature: polynucleotide sequence encoding the amino acid sequence of CDR1
ggccagtcagaacattggcacaagcatacagtg

SEQ ID NO: 26 - DNA, 21 bp, Artificial
  misc_feature: polynucleotide sequence encoding the amino acid sequence of CDR2
ttcttctgagtctatctctgg

SEQ ID NO: 27 - DNA, 27 bp, Artificial
  misc_feature: polynucleotide sequence encoding the amino acid sequence of CDR3
acaaagtaatacctggccattcacgtt

SEQ ID NO: 28 - DNA, 15 bp, Artificial
  misc_feature: polynucleotide sequence encoding the amino acid sequence of CDR1′
ttatattatccactg

SEQ ID NO: 29 - DNA, 51 bp, Artificial
  misc_feature: polynucleotide sequence encoding the amino acid sequence of CDR2′
ttttaatccttacaatcatggtactaagtacaatgagaagttcaaaggcag

SEQ ID NO: 30 - DNA, 27 bp, Artificial
  misc_feature: polynucleotide sequence encoding the amino acid sequence of CDR3′
aggaccctatgcctggtttgacacctg